(19) United States
(12) Patent Application Publication
(10) Pub. No.: US 2025/0325483 A1
(43) Pub. Date: Oct. 23, 2025

(54) FLOWPACK WITH COMPOSITION FOR IMPROVED TASTE MASKING OF ACTIVES

(71) Applicant: FERTIN PHARMA A/S, Vejle (DK)

(72) Inventors: Bruno Provstgaard Nielsen, Vejle (DK); Helle Martinussen, Vejle (DK); Helle Wittorff, Vejle (DK)

(21) Appl. No.: 18/876,619

(22) PCT Filed: Jun. 29, 2023
(86) PCT No.: PCT/DK2023/050172

§ 371 Date: Dec. 18, 2024

Related U.S. Application Data
(63) Continuation of application No. 17/854,794, filed on Jun. 30, 2022, now abandoned.

(30) Foreign Application Priority Data
Jun. 30, 2022 (CA) 3166262

(51) Int. Cl. A61K 9/16; A61K 9/00; A61K 45/06

(52) U.S. Cl. CPC A61K 9/1623; A61K 9/0056; A61K 9/1652; A61K 45/06

(57) ABSTRACT

A powder delivery system that is not in tablet form but contained in a flowpack. There is provided a flowpack for oral delivery of active pharmaceutical ingredients comprising a flowable population of particles including different types of particles where the particles comprise a first type of particles comprising one or more distinct portions of particles with a content of one or more active pharmaceutical ingredients and a carrier for the one or more active pharmaceutical ingredients, a second type of particles in an amount of 30-95% by weight of the population of particles separate from the first type of particles, the second type of particles comprising at least 95% by weight of one or more sweeteners, and a third type of particles in an amount of 1-10% by weight of the population of particles separate from the first and second type of particles, the third type of particles comprising flavor.

### CROSS-REFERENCE TO RELATED APPLICATIONS

This application is a 371 National Stage application of International Application No. PCT/DK2023/050172, filed Jun. 29, 2023, which claims priority to U.S. application Ser. No. 17/854,794, filed Jun. 30, 2022, and to Canadian Patent Application No. 3166262, filed Jun. 30, 2022.

### TECHNICAL FIELD

The present invention relates to the field of flowpacks comprising a powder system suitable for oral delivery of active ingredients. In particular, the invention relates to a powder delivery system suitable for administration of active pharmaceutical ingredients.

### BACKGROUND OF THE INVENTION

Taste masking of active pharmaceutical ingredients has been the focus of formulations of numerous oral administration forms in the prior art. While active pharmaceutical ingredients for gastrointestinal delivery are typically formulated as tablets without attention to taste masking, oral delivery of bitter tasting actives requires a different focus.

Typically, the challenge of bitter tasting actives for oral delivery has been solved in various ways that all serve to either delay the release of the actives or prevent the bitterness receptors of the oral cavity to direct exposure to the actives. Some of the solutions suggested involves certain carriers for actives, certain complexes, encapsulations, etc. Common to most of these methods of preventing exposure to bitterness receptors of the oral cavity is that these are intrinsically linked to formulation in a tablet dispension form.

Both in terms of convenience and compliance, oral tablets have certain benefits compared to other delivery vehicles for oral administration of active ingredients. Additional benefits include uniformity of content which is of particular importance for active pharmaceutical ingredients where lack of safety and appropriate delivery may become fatal in alleviating or treating medical conditions.

Oral tablets for oromucosal delivery of active ingredients are also commonly preferred with respect to securing an appropriate route of administration. Typically, such oral tablets are made by direct compression or compaction methods where a powder tablet material and an active ingredient are pressed into defined tablets with appropriate strength to provide a pharmacological effect to a patient in need thereof, in medical formulations, or to provide a health benefit for consumers in nutraceutical formulations.

Although the previous improvements of formulating oral tablets may benefit taste masking of active pharmaceutical ingredients to a certain degree, hitherto known oral tablets may be associated with various drawbacks. For instance, the time delay from administration of oral tablets to full efficacy of the active ingredient may inherently be delayed since the active ingredients are usually released over time from the oral tablet. Different improved tablets have been provided, such as oral disintegrating tablets where the aim is to have the tablets disintegrate relatively fast. However, these tablets may only help but do not solve the issue of delay and usually not adequately the problems with taste masking.

Accordingly, there is still a need for formulations that may help in obtaining improved sensorial properties of active ingredient delivery while at the same time providing fast release of active pharmaceutical ingredients in the oral cavity. Apart from taste masking, important sensorial properties include mouthfeel, melting sensation, flavor sensation, salivation, and cooling sensation associated with active ingredients. These properties are both relevant from a convenience perspective in oral administration, but certainly also in order to support an appropriate delivery of active ingredients and avoid adverse side effects of active ingredients. In particular mouthfeel is one of the more important sensorial properties of active ingredient delivery apart from efficacy and taste masking.

With respect to taste masking, the challenges are more profound with a higher release of such active ingredients. If off-notes are the predominant sensation during administration, convenience may be affected and even more critically, delivery of such active ingredients may also be affected. Hitherto, it has been a challenge to formulate an oral delivery platform that both serves to provide a very fast release of bitter active pharmaceutical ingredients and at the same time serves to provide excellent taste masking properties.

Additionally, only minor attention is given to benefits that may help obtaining release characteristics of active ingredients resulting in increased convenience and effectiveness. One of these release characteristics is increased generation of saliva. Increased generation of saliva and particularly an experience of increased saliva generation upon administration may for instance have some pronounced benefits for delivery of active ingredients to mucosal surfaces.

Hence, there is a need in the prior art for improved administration platforms that solve the above-referenced challenges and problems of the prior art. In particular, there is a need in the art for new platforms that support improved taste masking mouthfeel and convenience in combination with fast delivery of active ingredients.

### SUMMARY OF THE INVENTION

The present invention pertains to a new powder delivery system that is not in tablet form but contained in a flowpack. In particular, there is provided a flowpack for oral delivery of active pharmaceutical ingredients comprising a flowable population of particles including different types of particles where the particles comprise a first type of particles comprising one or more distinct portions of particles with a content of one or more active pharmaceutical ingredients and a carrier for the one or more active pharmaceutical ingredients, a second type of particles in an amount of 30-95% by weight of the population of particles separate from the first type of particles, the second type of particles comprising at least 95% by weight of one or more sweeteners, and a third type of particles in an amount of 1-10% by weight of the population of particles separate from the first and second type of particles, the third type of particles comprising flavor.

Specifically, the flowpack comprising the different type of particles of the present invention unlike orodispensable tablets may be associated with various benefits in terms of sensorial properties, such as taste masking properties, and various other properties, such as release properties. The flowable population of particles is designed to encompass a synergistic combination of different types of particles. Combined, the different types of particles serve to both deliver active pharmaceutical ingredients with improved effect and to accommodate various sensorial benefits compared to conventional oral tablets, including improved mouthfeel. Also, the powder system is aimed to be superior compared to simpler and less intricate powder systems available for administration of active ingredients.

Compared to other powder-based systems for oral delivery of active pharmaceutical ingredients containing only one type of particles, the present system of particles surprisingly may leverage from a synergistic effect of fast release of active pharmaceutical ingredients in combination with improved taste masking and general mouthfeel. This combination is difficult to obtain since the effects work in different directions which usually cannot be combined.

Typically, prior art powder-based systems are based on only one type of particles where the actives are prepared in granules with an inert carrier material or a sweetener based carrier material without additional sweetener particles in the formulation and additional flavor particles in the formulation. While these prior art formulations may benefit from improved uniformity of content of an active pharmaceutical ingredient, various drawbacks are associated with these formulations, such as inferior taste masking properties and mouthfeel, as well as inferior release of the actives.

The inventors of the present invention did not expect that combining the types of particles according to the invention would solve various of the prior art issues with oral tablets and more simple powder delivery systems. Such issues include improved saliva generation, appropriate delivery of active ingredients combined with beneficial sensorial properties, such as taste masking properties.

Particularly, the present invention may help in obtaining a release characteristic of active ingredients that offers increased convenience and effectiveness in combination with a pleasant taste and general liking of the products. One of these release characteristics is increased generation of saliva. Increased saliva generation and particularly an experience of increased saliva generation upon administration may for instance have some pronounced benefits for delivery of active ingredients to mucosal surfaces.

In some embodiments of the invention, the carrier of the first type of particles comprises an inert carrier, solely present as a result of the preparation of the active or to provide a support for the active. In some embodiments, the carrier of the first type of particles is exclusively based on an inert carrier, solely present as a result of the preparation of the active or to provide a support for the active.

In some other embodiments of the invention, the carrier of the first type of particles comprises a sweetener separate from the sweeteners of the second types of particles, resulting in granules with a composition of at least one active pharmaceutical ingredient, optionally supported by an inert carrier, and a sweetener granulated with the active pharmaceutical ingredient. In some other embodiments of the invention, the carrier of the first type of particles is exclusively based on a sweetener separate from the sweeteners of the second types of particles, resulting in granules with a composition of at least one active pharmaceutical ingredient, optionally supported by an inert carrier, and a sweetener granulated with the active pharmaceutical ingredient.

In the present context, the expression “granulation”, “granulated” or “granules”, when mentioned in context with the first types of particles, is intended to mean that the active pharmaceutical ingredients have been subject of a granulation process as commonly applied in the industry. This may involve dry or wet granulation or similar processes.

In the present context, the expression “separate from” is intended to mean that the types of particles of the present invention are not prepared in the same process but are located as separate types of particles in the formulation for use in the flowpack. These types of particles may to some degree agglomerate together under certain conditions during storage or in the mixing process of the formulation but this is not the aim of the present invention. The aim is that the different types of particles are located as a flowable population of particles and that the types of particles are dispersed together in the powder blend.

For instance, the first type of particles comprises the active pharmaceutical ingredients, whereas the second type of particles does not comprise an active pharmaceutical ingredient but substantially solely sweeteners, and for the third type of particles substantially solely flavors. In the present context, flavors are not intended to have a function as active pharmaceutical ingredients. Accordingly, the present invention comprises at least three type of particles that are structurally distinguishable from each other, separated from each other, having distinct properties in the final blend of flowable population of particles.

The synergy of having at least three types of particles of the present invention may help in obtaining improved sensorial properties of active ingredient delivery. Here, important sensorial properties include mouthfeel, melting sensation, flavor sensation, salivation, cooling sensation, and off-note sensation associated with active ingredients or processing aids. Of particular concern is to provide a suitable mouthfeel in order to allow medical patients or consumers seeking health benefits a more accommodating treatment or alleviation of symptoms. Also, the present invention may help in improving taste-masking of off-notes during administration. The taste masking challenge is more profound when a higher release of such active ingredients is provided which is generally the case for the system of the present invention.

The unique combination of the types of particles according to the invention may provide additional advantages that conventional powder systems may not provide. One of such advantages is improved mouthfeel. Another is improved generation of saliva. Yet another is improved cooling sensation. Other advantages may include improved melting sensation, flavor sensation, and off-note sensation associated with active ingredients.

The special property of “mouthfeel” involves various factors of the population of particles that combined contribute to the overall impression of mouthfeel. Objective criteria are set up for test panels that evaluate mouthfeel according to the invention. Among these criteria may be elements such as roughness impression, texture impression such as a sandy impression and/or a melting impression. The general aim of the invention may be to improve these elements to obtain an improved mouthfeel of the population of particles. Combined with improved release and improved taste masking as mentioned above, the invention may ascertain synergistic benefits compared to conventional tablets or more simple powder delivery systems known in the art.

In the present context, when the population of particles is mentioned to be “dry and flowable”, the intended meaning is that the system behaves as a powder in the way that the water content is suitably low for a skilled person within powder technology to consider it “dry” for the purpose of the invention and being able to “flow” for a skilled person within powder technology to consider it “flowable” for the purpose of the invention. For instance, the system does not need to have a certain water content of 0.0% but may have a content of water to a degree that it behaves like a powder for the purpose of the invention, such as less than 10.0% by weight, 8.0% by weight, 6.0% by weight, 4.0% by weight, 2.0% by weight water content, 1.5% by weight water, such as less than 1.0% by weight water content.

In terms of being “flowable”, the population of particles according to the invention in some embodiments does not need to be “free-flowing”. In some embodiments it is adequate that the powder is able to “flow” in the sense that certain agglomerations of particles are allowed and that not all types of particles in the population of particles is to be free-flowing. For instance, agglomeration to some extent during storage may be allowed, just that the powder may be gently handled to make the powder flowable to some extent. For the avoidance of doubt, a tablet is not considered “flowable” in the present context. Hence, the population of particles according to the invention is not a tablet or comprised in a tablet, such as a chewable tablet or orally disintegrating tablet according to the invention.

In some embodiments of the invention, the flowable population of particles is a free-flowing population of particles.

In some embodiments of the invention, the population of particles is a dry and substantially free-flowing population of particles.

In some embodiments of the invention, the flowable population of particles constitutes more than 70% by weight of the flowpack. In some embodiments of the invention, the flowable population of particles constitutes more than 80% by weight of the flowpack. In some embodiments of the invention, the flowable population of particles constitutes more than 90% by weight of the flowpack. In some embodiments of the invention, the flowable population of particles constitutes more than 95% by weight of the flowpack. In some embodiments of the invention, the flowable population of particles constitutes more than 99% by weight of the flowpack. In some embodiments of the invention, the flowable population of particles constitutes 100% by weight of the flowpack. In this context, it is understood that the package material is not included in the percentage.

In some embodiments of the invention, the first, second and third type of particles constitutes more than 70% by weight of the flowable population of particles. In some embodiments of the invention, the first, second and third type of particles constitutes more than 80% by weight of the flowable population of particles. In some embodiments of the invention, the first, second and third type of particles constitutes more than 90% by weight of the flowable population of particles. In some embodiments of the invention, the first, second and third type of particles constitutes more than 95% by weight of the flowable population of particles. In some embodiments of the invention, the first, second and third type of particles constitutes more than 99% by weight of the flowable population of particles. In some embodiments of the invention, the first, second and third type of particles constitutes 100% by weight of the flowable population of particles.

In some embodiments of the invention, the second type of particles is present in an amount of 35-95% by weight of the population of particles. In some embodiments of the invention, the second type of particles is present in an amount of 35-90% by weight of the population of particles. In some embodiments of the invention, the second type of particles is present in an amount of 35-85% by weight of the population of particles. In some embodiments of the invention, the second type of particles is present in an amount of 35-80% by weight of the population of particles.

In some embodiments of the invention, the second type of particles is present in an amount of 40-80% by weight of the population of particles. In some embodiments of the invention, the second type of particles is present in an amount of 40-70% by weight of the population of particles. In some embodiments of the invention, the second type of particles is present in an amount of 40-60% by weight of the population of particles.

In some embodiments of the invention, the second type of particles is present in an amount of 20-50% by weight of the population of particles. In some embodiments of the invention, the second type of particles is present in an amount of 20-45% by weight of the population of particles. In some embodiments of the invention, the second type of particles is present in an amount of 20-40% by weight of the population of particles.

In some embodiments of the invention, an average particle size of the second type of particles is between 50 and 500 microns.

In some embodiments of the invention, an average particle size of the second type of particles is between 100 and 400 microns.

A particular advantage of the present invention is achieved when the different types of particles have an average particle size that is similar or the about the same. An even more particular advantage of the present invention is achieved when the different types of particles have an average particle size that is similar or the about the same together with a distribution of particles that are similar or about the same.

In some embodiments of the invention, the first and second types of particles have an average particle size that is similar or the about the same. In some embodiments of the invention, the first and second types of particles have an average particle size that is similar or the about the same together with a distribution of particles that are similar or about the same.

In some embodiments of the invention, the first and third types of particles have an average particle size that is similar or the about the same. In some embodiments of the invention, the first and third types of particles have an average particle size that is similar or the about the same together with a distribution of particles that are similar or about the same.

In some embodiments of the invention, the second and third types of particles have an average particle size that is similar or the about the same. In some embodiments of the invention, the second and third types of particles have an average particle size that is similar or the about the same together with a distribution of particles that are similar or about the same.

In some embodiments of the invention, the first, second and third types of particles have an average particle size that is similar or the about the same. In some embodiments of the invention, the first, second and third types of particles have an average particle size that is similar or the about the same together with a distribution of particles that are similar or about the same.

In some embodiments of the invention, a series of at least 5 samples of the second type of particles each having the same fixed weight in the range of 0.5-2 g varying with a relative standard deviation (RSD) below 10%.

Generally, the method used for content uniformity of samples is determined according to European Pharmacopoeia 10.8 when using test method 2.9.40. Uniformity of dosage units. The acceptance value (AV) is calculated using mass variation (MV) or content uniformity (CU) depending on the dose and ratio of the drug substance. An appropriate analytical method is selected for content uniformity.

Particularly when the different ingredients have different particle sizes, segregation may lead to different contents of content of uniformity. Yet, another aspect is that even storing a thoroughly mixed composition for too long may lead to segregation. In is noted that the term segregation as used herein would be known to the skilled person to mean the separation of a mixture according to similarity, typically size. This may in the present context be a problem when handling a mixture comprising very different sizes of particles.

In some embodiments of the invention, a series of at least 5 samples of the second type of particles each having the same fixed weight in the range of 0.5-2 g varying with a relative standard deviation (RSD) below 5%.

In some embodiments of the invention, the second type of particles comprises at least 98% by weight of one or more sweeteners.

In some embodiments of the invention, the one or more sweeteners of the second type of particles comprises directly compressible (DC) sweetener particles.

The term “DC sweetener particles” refers to particles of direct compressible (DC) sweetener. It is noted that the terms “DC sweetener particles” and “DC particles” are used interchangeably. DC sweetener particles may be obtained by granulating non-DC sweetener with e.g. other sweeteners or binders for the purpose of obtaining so-called direct compressible particles (DC). This may be done in a process such as a wet granulation process, or in a dry granulation process. Also, granulation of non-DC sweetener with water as binder is considered to result in DC sweetener particles in the present context. Agglomeration of particles into a single particle is also within the intended meaning. Typically, DC sweetener particles have a surface morphology with a rough surface morphology when seen in a scanning electron microscope. In the present context, “granulation” or “granulated” or “agglomeration” or similar wording is not intended to involve milling, comminuting, or grinding of larger crystalline particles into smaller particles.

The term “DC sweetener particles that are not granulated sweetener particles” refers to particles of direct compressible (DC) sweetener, which have not been granulated but are DC by nature. Sorbitol particles is an example of such particles. Dextrose that has not been granulated is also considered an example of such particles. In the present context, “granulation” or “granulated” or similar wording is not intended to involve milling, comminuting, or grinding of larger crystalline particles into smaller particles.

In some embodiments of the invention, the one or more sweeteners of the second type of particles comprises non-directly compressible (non-DC) sweetener particles.

The term “non-DC sugar alcohol particles” refers to particles of non-directly compressible (non-DC) sweetener. It is noted that the terms “non-DC sweetener particles” and “non-DC particles” are used interchangeably. In the present context, the non-DC sweetener particles refer to particles which have not been preprocessed by granulation with e.g. other sweeteners or binders for the purpose of obtaining so-called direct compressible particles (DC). Typically, non-DC sweetener particles include particles obtained by crystallization, optionally followed by milling, comminuting, or grinding, which does not involve other sweeteners or binders. Thus, non-DC sweetener particles are considered as particles consisting of non-DC sweetener. However, some degree of impurity may be present. Hence, the particles may be considered as substantially consisting of non-DC sweetener. Likewise, non-DC sweetener particles are considered as non-granulated sweetener particles. Typically, non-DC sweetener particles have a surface morphology with a smooth surface morphology when seen in a scanning electron microscope compared to DC sweetener particles.

In one embodiment of the invention, non-DC sweetener particles include crystalline sweetener particles obtained by a crystallization process, optionally followed by milling, comminuting, or grinding, which does not involve other sweeteners or binders. In this context, “crystalline” is intended to mean that the individual particles are composed of a coherent crystal structure and not for instance a micro-crystalline structure where small crystalline particles are gathered to larger particles.

One advantage of the invention is a surprisingly strong saliva generation compared to conventional formulations. Particularly, the non-DC particles surprisingly induce a remarkable generation of saliva. Increased generation of saliva may have a huge impact on the delivery of the one or more active ingredients. Specifically, increased generation of saliva may increase exposure of the one or more active ingredients to mucosal surfaces and thereby contribute to an increased uptake in the oral mucosa. More specifically, when increased generation of saliva is commenced in a short time, the one or more active ingredients may relatively quickly be exposed to mucosal surfaces and thereby relatively quickly deliver a desired effect. Hence, a synergy between uptake of active ingredients and increased saliva generation may be seen according to the invention. Non-DC erythritol is an example of a sugar alcohol that may contribute significantly to increased generation of saliva.

One unexpected advantage over the prior art is that the saliva generation is surprisingly sustained even after a user has swallowed the bulk-portion of the non-DC sweeteners. This sustaining of the salivation generation may be advantageous in relation to many applications of the formulation ranging from mouthfeel, taste, flavor perception, etc.

In some embodiments of the invention, the one or more sweeteners of the second type of particles comprises non-directly compressible (non-DC) sweetener particles and directly compressible (DC) sweetener particles in a ratio of 0.2 and 1.2.

In some embodiments of the invention, the one or more sweeteners of the second type of particles comprises non-directly compressible (non-DC) sweetener particles and directly compressible (DC) sweetener particles in a ratio of 0.3 and 0.7.

In some embodiments of the invention, the one or more sweeteners of the second type of particles comprises one or more sugar alcohols.

In some embodiments of the invention, the one or more sweeteners of the second type of particles comprises one or more sugar alcohols selected from the group consisting of erythritol, maltitol, xylitol, isomalt, lactitol, mannitol, sorbitol, and combinations thereof.

In some embodiments of the invention, the one or more sweeteners of the second type of particles comprises one or more sugar alcohols selected from the group consisting of erythritol, maltitol, xylitol, isomalt, mannitol, and combinations thereof.

In some embodiments of the invention, the one or more sweeteners of the second type of particles comprises one or more sugar alcohols selected from the group consisting of erythritol, maltitol, xylitol, isomalt, lactitol, and combinations thereof.

In some embodiments of the invention, the one or more sweeteners of the second type of particles comprises one or more sugar alcohols selected from the group consisting of erythritol, maltitol, xylitol, isomalt, and combinations thereof.

In some embodiments of the invention, the one or more sweeteners of the second type of particles comprises one or more sugar alcohols selected from the group consisting of erythritol, maltitol, xylitol, and combinations thereof.

In some embodiments of the invention, the one or more sweeteners of the second type of particles comprises erythritol. In some embodiments of the invention, the one or more sweeteners of the second type of particles comprises maltitol. In some embodiments of the invention, the one or more sweeteners of the second type of particles comprises xylitol. In some embodiments of the invention, the one or more sweeteners of the second type of particles comprises isomalt. In some embodiments of the invention, the one or more sweeteners of the second type of particles comprises lactitol. In some embodiments of the invention, the one or more sweeteners of the second type of particles comprises mannitol.

These may be available in a non-DC form of the relevant sugar alcohol as particles which have not been preprocessed by granulation with other sugar alcohols or binders for the purpose of obtaining so-called direct compressible particles (DC) on the basis of sugar alcohol particles which are by themselves not suitable for direct compression. Such non-DC particles of sugar alcohol may typically consist of the sugar alcohol. Therefore, non-DC sugar alcohol particles may typically be particles consisting of sugar alcohol, which is non-directly compressible in its pure form. Or they may be present in a DC form.

It should be noted that the terminology non-DC is easily understood within the field of technology. Suppliers or sugar alcohol provides clear guidance to the user as for the ability for use in relation to compression of tablets. A non-DC particle in this connection is referred to as a particle which is not expressly recommended by the supplier for compression. Examples of a non-DC grade of erythritol includes Zerose™ erythritol 16952F supplied by Cargill. Further examples of non-DC sugar alcohol particles include non-DC xylitol as Xivia C from Dupont, non-DC isomalt as Isomalt GS from Beneo Paltinit, non-DC mannitol as C*PharmMannidex 16700 from Cargill, non-DC maltitol as Maltisorb P200 from Roquette.

Non-direct compressible (non-DC) sugar alcohols may include non-DC grades of Xylitol, non-DC grades of Erythritol, non-DC grades of Mannitol, non-DC grades of maltitol, non-DC grades of Lactitol, non-DC grades of Isomalt, or other suitable non-DC grades of sugar alcohols.

In some embodiments of the invention, the one or more sweeteners of the second type of particles comprises one or more saccharides.

In some embodiments of the invention, the one or more sweeteners of the second type of particles comprises one or more saccharides selected from the group consisting of dextrose, saccharose, dextrin, trehalose, fructose, and combinations thereof.

In some embodiments of the invention, the one or more sweeteners of the second type of particles comprises dextrose. In some embodiments of the invention, the one or more sweeteners of the second type of particles comprises saccharose. In some embodiments of the invention, the one or more sweeteners of the second type of particles comprises dextrin. In some embodiments of the invention, the one or more sweeteners of the second type of particles comprises trehalose. In some embodiments of the invention, the one or more sweeteners of the second type of particles comprises fructose.

In some embodiments of the invention, the third type of particles is present in an amount of 1-8% by weight of the population of particles.

In some embodiments of the invention, the third type of particles is present in an amount of 1-5% by weight of the population of particles.

In some embodiments of the invention, the third type of particles is present in an amount of 2-5% by weight of the population of particles.

In some embodiments of the invention, an average particle size of the third type of particles is between 50 and 500 microns.

In some embodiments of the invention, an average particle size of the third type of particles is between 100 and 400 microns.

In some embodiments of the invention, a series of at least 5 samples of the third type of particles each having the same fixed weight in the range of 0.5-2 g varying with a relative standard deviation (RSD) below 10%.

Generally, the method used for content uniformity of samples is determined according to European Pharmacopoeia 10.8 when using test method 2.9.40. Uniformity of dosage units. The acceptance value (AV) is calculated using mass variation (MV) or content uniformity (CU) depending on the dose and ratio of the drug substance. An appropriate analytical method is selected for content uniformity.

Particularly when the different ingredients have different particle sizes, segregation may lead to different contents of content of uniformity. Yet, another aspect is that even storing a thoroughly mixed composition for too long may lead to segregation. In is noted that the term segregation as used herein would be known to the skilled person to mean the separation of a mixture according to similarity, typically size. This may in the present context be a problem when handling a mixture comprising very different sizes of particles.

In some embodiments of the invention, a series of at least 5 samples of the third type of particles each having the same fixed weight in the range of 0.5-2 g varying with a relative standard deviation (RSD) below 5%.

In some embodiments of the invention, the third type of particles comprises a powder flavor.

In some embodiments of the invention, the third type of particles is a powder flavor.

In some embodiments of the invention, the third type of particles has a load of flavor in a range of 5-25%.

In some embodiments of the invention, the flavor of the third type of particles is selected from the group consisting of coconut, coffee, vanilla, blackcurrant, lemon, grapefruit, orange, lime, menthol, liquorice, caramel aroma, honey aroma, pineapple, strawberry, raspberry, tropical fruits, cherries, cinnamon, peppermint, wintergreen, spearmint, eucalyptus, mint, fruit essence, creme essence, and combinations thereof.

Further usable flavors include almond, almond amaretto, apple, Bavarian cream, black cherry, black sesame seed, blueberry, brown sugar, bubblegum, butterscotch, cappuccino, caramel, caramel cappuccino, cheesecake (graham crust), chili, cinnamon redhots, cotton candy, circus cotton candy, clove, coconut, coffee, clear coffee, double chocolate, energy cow, ginger, glutamate, graham cracker, grape juice, green apple, Hawaiian punch, honey, Jamaican rum, Kentucky bourbon, kiwi, koolada, lemon, lemon lime, tobacco, maple syrup, maraschino cherry, marshmallow, menthol, milk chocolate, mocha, Mountain Dew, peanut butter, pecan, peppermint, raspberry, banana, ripe banana, root beer, RY 4, spearmint, strawberry, sweet cream, sweet tarts, sweetener, toasted almond, tobacco, tobacco blend, vanilla bean ice cream, vanilla cupcake, vanilla swirl, vanillin, waffle, Belgian waffle, watermelon, whipped cream, white chocolate, wintergreen, amaretto, banana cream, black walnut, blackberry, butter, butter rum, cherry, chocolate hazelnut, cinnamon roll, cola, creme de menthe, eggnog, English toffee, guava, lemonade, licorice, maple, mint chocolate chip, orange cream, peach, pina colada, pineapple, plum, pomegranate, pralines and cream, red licorice, salt water taffy, strawberry banana, strawberry kiwi, tropical punch, tutti frutti, vanilla, or any combination thereof.

In some embodiments of the invention, one or more distinct portions of particles of the first type of particles is present in an amount of 30-60% by weight of the population of particles.

In some embodiments of the invention, one or more distinct portions of particles of the first type of particles is present in an amount of 40-60% by weight of the population of particles.

In some embodiments of the invention, one or more distinct portions of particles of the first type of particles is present in an amount of 10-40% by weight of the population of particles.

In some embodiments of the invention, one or more distinct portions of particles of the first type of particles is present in an amount of 20-40% by weight of the population of particles.

In some embodiments of the invention, one or more distinct portions of particles of the first type of particles is present in an amount of 2-20% by weight of the population of particles.

In some embodiments of the invention, one or more distinct portions of particles of the first type of particles is present in an amount of 2-10% by weight of the population of particles.

In some embodiments of the invention, an average particle size of the first type of particles is between 50 and 500 microns.

In some embodiments of the invention, an average particle size of the first type of particles is between 100 and 400 microns.

In some embodiments of the invention, an average particle size of the first type of particles is between 200 and 400 microns.

In some embodiments of the invention, an average particle size of the first type of particles is between 400 and 600 microns.

In some embodiments of the invention, a series of at least 5 samples of the first type of particles each having the same fixed weight in the range of 0.5-2 g varying with a relative standard deviation (RSD) below 10%.

Generally, the method used for content uniformity of samples is determined according to European Pharmacopoeia 10.8 when using test method 2.9.40. Uniformity of dosage units. The acceptance value (AV) is calculated using mass variation (MV) or content uniformity (CU) depending on the dose and ratio of the drug substance. An appropriate analytical method is selected for content uniformity.

Particularly when the different ingredients have different particle sizes, segregation may lead to different contents of content of uniformity. Yet, another aspect is that even storing a thoroughly mixed composition for too long may lead to segregation. In is noted that the term segregation as used herein would be known to the skilled person to mean the separation of a mixture according to similarity, typically size. This may in the present context be a problem when handling a mixture comprising very different sizes of particles.

In some embodiments of the invention, a series of at least 5 samples of the first type of particles each having the same fixed weight in the range of 0.5-2 g varying with a relative standard deviation (RSD) below 5%.

In some embodiments of the invention, the one or more distinct portions of particles of the first type of particles comprises one or more low dose active pharmaceutical ingredients.

In some embodiments of the invention, the one or more distinct portions of particles of the first type of particles comprises one or more low dose active pharmaceutical ingredients in an amount of less than 4% by weight of the population of particles. In some embodiments of the invention, the one or more distinct portions of particles of the first type of particles comprises one or more low dose active pharmaceutical ingredients in an amount of less than 3% by weight of the population of particles. In some embodiments of the invention, the one or more distinct portions of particles of the first type of particles comprises one or more low dose active pharmaceutical ingredients in an amount of less than 2% by weight of the population of particles. In some embodiments of the invention, the one or more distinct portions of particles of the first type of particles comprises one or more low dose active pharmaceutical ingredients in an amount of less than 1% by weight of the population of particles.

In some embodiments of the invention, the one or more distinct portions of particles of the first type of particles comprises one or more low dose active pharmaceutical ingredients selected from the group consisting of diphenhydramine, cetirizine, loratadine, chlorpheniramine maleate, levocetirizine, meclizine, dextromethorphan, phenylephrine, famotidine, omeprazole, doxylamine succinate, melatonin, and any combination and mixture thereof.

In some embodiments of the invention, the one or more active pharmaceutical ingredients comprises diphenhydramine. In some embodiments of the invention, the one or more active pharmaceutical ingredients comprises cetirizine. In some embodiments of the invention, the one or more active pharmaceutical ingredients comprises loratadine. In some embodiments of the invention, the one or more active pharmaceutical ingredients comprises chlorpheniramine maleate. In some embodiments of the invention, the one or more active pharmaceutical ingredients comprises chlorpheniramine levocetirizine. In some embodiments of the invention, the one or more active pharmaceutical ingredients comprises meclizine. In some embodiments of the invention, the one or more active pharmaceutical ingredients comprises dextromethorphan. In some embodiments of the invention, the one or more active pharmaceutical ingredients comprises phenylephrine. In some embodiments of the invention, the one or more active pharmaceutical ingredients comprises famotidine. In some embodiments of the invention, the one or more active pharmaceutical ingredients comprises omeprazole. In some embodiments of the invention, the one or more active pharmaceutical ingredients comprises doxylamine succinate. In some embodiments of the invention, the one or more active pharmaceutical ingredients comprises melatonin.

In some embodiments of the invention, the one or more distinct portions of particles of the first type of particles comprises one or more low dose antihistamine ingredients selected from the group consisting of diphenhydramine, cetirizine, loratadine, chlorpheniramine maleate, levocetirizine, and any combination and mixture thereof.

In some embodiments of the invention, the one or more distinct portions of particles of the first type of particles comprises one or more low dose cough suppressant ingredients comprising dextromethorphan.

In some embodiments of the invention, the one or more distinct portions of particles of the first type of particles comprises one or more low dose motion sickness ingredients comprising meclizine.

In some embodiments of the invention, the one or more distinct portions of particles of the first type of particles comprises one or more low dose decongestant ingredients comprising phenylephrine.

In some embodiments of the invention, the one or more distinct portions of particles of the first type of particles comprises one or more low dose heartburn ingredients selected from the group consisting of famotidine, omeprazole, and any combination and mixture thereof.

In some embodiments of the invention, the one or more distinct portions of particles of the first type of particles comprises one or more low dose insomnia ingredients selected from the group consisting of doxylamine succinate, melatonin, and any combination and mixture thereof.

In some embodiments of the invention, the one or more distinct portions of particles of the first type of particles comprises one or more low dose active pharmaceutical ingredients present in an amount of 2-30 mg in the flow pack. In some embodiments of the invention, the one or more distinct portions of particles of the first type of particles comprises one or more low dose active pharmaceutical ingredients present in an amount of 2-20 mg in the flow pack. In some embodiments of the invention, the one or more distinct portions of particles of the first type of particles comprises one or more low dose active pharmaceutical ingredients present in an amount of 2-10 mg in the flow pack.

In some embodiments of the invention, the one or more distinct portions of particles of the first type of particles comprising the one or more low dose active pharmaceutical ingredients is present in an amount of 2-20% by weight of the population of particles.

In some embodiments of the invention, the one or more distinct portions of particles of the first type of particles comprising the one or more low dose active pharmaceutical ingredients is present in an amount of 2-10% by weight of the population of particles.

In some embodiments of the invention, the one or more distinct portions of particles of the first type of particles comprises one or more medium dose active pharmaceutical ingredients.

In some embodiments of the invention, the one or more distinct portions of particles of the first type of particles comprises one or more medium dose active pharmaceutical ingredients in an amount of 4 to 30% by weight of the population of particles.

In some embodiments of the invention, the one or more distinct portions of particles of the first type of particles comprises one or more medium dose active pharmaceutical ingredients in an amount of 4 to 20% by weight of the population of particles.

In some embodiments of the invention, the one or more distinct portions of particles of the first type of particles comprises one or more medium dose active pharmaceutical ingredients in an amount of 2 to 20% by weight of the population of particles.

In some embodiments of the invention, the one or more active pharmaceutical ingredients comprises one or more medium dose active pharmaceutical ingredients in an amount of more than 2% by weight of the population of particles. In some embodiments of the invention, the one or more active pharmaceutical ingredients comprises one or more medium dose active pharmaceutical ingredients in an amount of more than 3% by weight of the population of particles. In some embodiments of the invention, the one or more active pharmaceutical ingredients comprises one or more medium dose active pharmaceutical ingredients in an amount of more than 4% by weight of the population of particles. In some embodiments of the invention, the one or more active pharmaceutical ingredients comprises one or more medium dose active pharmaceutical ingredients in an amount of more than 5% by weight of the population of particles.

In some embodiments of the invention, the one or more active pharmaceutical ingredients comprises one or more medium dose active pharmaceutical ingredients in an amount of less than 30% by weight of the population of particles. In some embodiments of the invention, the one or more active pharmaceutical ingredients comprises one or more medium dose active pharmaceutical ingredients in an amount of less than 25% by weight of the population of particles. In some embodiments of the invention, the one or more active pharmaceutical ingredients comprises one or more medium dose active pharmaceutical ingredients in an amount of less than 20% by weight of the population of particles. In some embodiments of the invention, the one or more active pharmaceutical ingredients comprises one or more medium dose active pharmaceutical ingredients in an amount of less than 15% by weight of the population of particles.

In some embodiments of the invention, the one or more distinct portions of particles of the first type of particles comprises one or more medium dose active pharmaceutical ingredients selected from the group consisting of diphenhydramine, fexofenadine, dimenhydrinate, meclizine, pseudoephedrine, aspirin, caffeine, theanine, and any combination and mixture thereof.

In some embodiments of the invention, the one or more active pharmaceutical ingredients comprises diphenhydramine. In some embodiments of the invention, the one or more active pharmaceutical ingredients comprises fexofenadine. In some embodiments of the invention, the one or more active pharmaceutical ingredients comprises dimenhydrinate. In some embodiments of the invention, the one or more active pharmaceutical ingredients comprises meclizine. In some embodiments of the invention, the one or more active pharmaceutical ingredients comprises pseudoephedrine. In some embodiments of the invention, the one or more active pharmaceutical ingredients comprises aspirin. In some embodiments of the invention, the one or more active pharmaceutical ingredients comprises caffeine. In some embodiments of the invention, the one or more active pharmaceutical ingredients comprises theanine.

In some embodiments of the invention, the one or more distinct portions of particles of the first type of particles comprises one or more medium dose antihistamine ingredients selected from the group consisting of diphenhydramine, fexofenadine, and any combination and mixture thereof.

In some embodiments of the invention, the one or more distinct portions of particles of the first type of particles comprises one or more medium dose motion sickness ingredients selected from the group consisting of dimenhydrinate, meclizine, and any combination and mixture thereof.

In some embodiments of the invention, the one or more distinct portions of particles of the first type of particles comprises one or more medium dose decongestant ingredients comprising pseudoephedrine.

In some embodiments of the invention, the one or more distinct portions of particles of the first type of particles comprises one or more medium dose analgesics comprising aspirin.

In some embodiments of the invention, the one or more distinct portions of particles of the first type of particles comprises one or more medium dose alertness ingredients selected from the group consisting of caffeine, theanine, and any combination and mixture thereof.

In some embodiments of the invention, the one or more distinct portions of particles of the first type of particles comprises one or more medium dose active pharmaceutical ingredients in an amount of 20-150 mg in the flow pack.

In some embodiments of the invention, the one or more distinct portions of particles of the first type of particles comprises one or more medium dose active pharmaceutical ingredients in an amount of 30-100 mg in the flow pack.

In some embodiments of the invention, the one or more distinct portions of particles of the first type of particles comprising the one or more medium dose active pharmaceutical ingredients is present in an amount of 10-40% by weight of the population of particles.

In some embodiments of the invention, the one or more distinct portions of particles of the first type of particles comprising the one or more medium dose active pharmaceutical ingredients is present in an amount of 20-40% by weight of the population of particles.

In some embodiments of the invention, the one or more distinct portions of particles of the first type of particles comprises one or more high dose active pharmaceutical ingredients.

In some embodiments of the invention, the one or more distinct portions of particles of the first type of particles comprises one or more high dose active pharmaceutical ingredients in an amount of 20 to 70% by weight of the population of particles. In some embodiments of the invention, the one or more distinct portions of particles of the first type of particles comprises one or more high dose active pharmaceutical ingredients in an amount of 20 to 60% by weight of the population of particles. In some embodiments of the invention, the one or more distinct portions of particles of the first type of particles comprises one or more high dose active pharmaceutical ingredients in an amount of 20 to 50% by weight of the population of particles. In some embodiments of the invention, the one or more distinct portions of particles of the first type of particles comprises one or more high dose active pharmaceutical ingredients in an amount of 20 to 40% by weight of the population of particles.

In some embodiments of the invention, the one or more distinct portions of particles of the first type of particles comprises one or more high dose active pharmaceutical ingredients selected from the group consisting of fexofenadine, guaifenesin, calcium carbonate, magnesium hydroxide, acetaminophen, aspirin, ibuprofen, naproxen sodium, caffeine, and any combination and mixture thereof.

In some embodiments of the invention, the one or more active pharmaceutical ingredients comprises fexofenadine. In some embodiments of the invention, the one or more active pharmaceutical ingredients comprises fexofenadine. In some embodiments of the invention, the one or more active pharmaceutical ingredients comprises fexofenadine. In some embodiments of the invention, the one or more active pharmaceutical ingredients comprises guaifenesin. In some embodiments of the invention, the one or more active pharmaceutical ingredients comprises calcium carbonate. In some embodiments of the invention, the one or more active pharmaceutical ingredients comprises magnesium hydroxide. In some embodiments of the invention, the one or more active pharmaceutical ingredients comprises acetaminophen. In some embodiments of the invention, the one or more active pharmaceutical ingredients comprises aspirin. In some embodiments of the invention, the one or more active pharmaceutical ingredients comprises ibuprofen. In some embodiments of the invention, the one or more active pharmaceutical ingredients comprises naproxen sodium. In some embodiments of the invention, the one or more active pharmaceutical ingredients comprises caffeine.

In some embodiments of the invention, the one or more distinct portions of particles of the first type of particles comprises one or more high dose antihistamine ingredients comprising fexofenadine.

In some embodiments of the invention, the one or more distinct portions of particles of the first type of particles comprises one or more high dose expectorant ingredients comprising guaifenesin.

In some embodiments of the invention, the one or more distinct portions of particles of the first type of particles comprises one or more high dose antacid ingredients selected from the group consisting of calcium carbonate, magnesium hydroxide, and any combination and mixture thereof.

In some embodiments of the invention, the one or more distinct portions of particles of the first type of particles comprises one or more high dose analgesics ingredients selected from the group consisting of acetaminophen, aspirin, ibuprofen, naproxen sodium, and any combination and mixture thereof.

In some embodiments of the invention, the one or more distinct portions of particles of the first type of particles comprises one or more high dose alertness ingredients comprising caffeine.

In some embodiments of the invention, the one or more distinct portions of particles of the first type of particles comprises one or more high dose active pharmaceutical ingredients in an amount of 150-1000 mg in the flow pack.

In some embodiments of the invention, the one or more distinct portions of particles of the first type of particles comprises one or more high dose active pharmaceutical ingredients in an amount of 250-600 mg in the flow pack.

In some embodiments of the invention, the one or more distinct portions of particles of the first type of particles comprising the one or more high dose active pharmaceutical ingredients is present in an amount of 30-70% by weight of the population of particles.

In some embodiments of the invention, the one or more distinct portions of particles of the first type of particles comprising the one or more high dose active pharmaceutical ingredients is present in an amount of 30-60% by weight of the population of particles.

In some embodiments of the invention, the one or more distinct portions of particles of the first type of particles comprising the one or more high dose active pharmaceutical ingredients is present in an amount of 30-50% by weight of the population of particles.

In some embodiments of the invention, the one or more distinct portions of particles of the first type of particles comprising the one or more high dose active pharmaceutical ingredients is present in an amount of 30-60% by weight of the population of particles.

In some embodiments of the invention, the one or more distinct portions of particles of the first type of particles comprising the one or more high dose active pharmaceutical ingredients is present in an amount of 40-60% by weight of the population of particles.

In some embodiments of the invention, a series of at least 5 samples of the population of particles each having the same fixed weight in the range of 0.5-2 g comprises the one or more active pharmaceutical ingredients in an amount varying with a relative standard deviation (RSD) below 10%.

Generally, the method used for content uniformity of samples is determined according to European Pharmacopoeia 10.8 when using test method 2.9.40. Uniformity of dosage units. The acceptance value (AV) is calculated using mass variation (MV) or content uniformity (CU) depending on the dose and ratio of the drug substance. An appropriate analytical method is selected for content uniformity.

Particularly when the different ingredients have different particle sizes, segregation may lead to different contents of content of uniformity. Yet, another aspect is that even storing a thoroughly mixed composition for too long may lead to segregation. In is noted that the term segregation as used herein would be known to the skilled person to mean the separation of a mixture according to similarity, typically size. This may in the present context be a problem when handling a mixture comprising very different sizes of particles.

In some embodiments of the invention, a series of at least 5 samples of the population of particles each having the same fixed weight in the range of 0.5-2 g comprises the one or more active pharmaceutical ingredients in an amount varying with a relative standard deviation (RSD) below 5%.

In some embodiments of the invention, a series of at least 5 samples of the population of particles each having the same fixed weight in the range of 0.5-2 g comprises the one or more active pharmaceutical ingredients in an amount varying with a relative standard deviation (RSD) below 2%.

In some embodiments of the invention, the first, second and third type of particles have an average particle size difference of at most 200 microns. In some embodiments of the invention, the first, second and third type of particles have an average particle size difference of at most 150 microns. In some embodiments of the invention, the first, second and third type of particles have an average particle size difference of at most 100 microns. In some embodiments of the invention, the first, second and third type of particles have an average particle size difference of at most 75 microns. In some embodiments of the invention, the first, second and third type of particles have an average particle size difference of at most 50 microns. In some embodiments of the invention, the first, second and third type of particles have an average particle size difference of at most 25 microns.

In some embodiments of the invention, the one or more distinct portions of particles of the first type of particles have an average particle size difference of at most 100 microns.

In some embodiments of the invention, the one or more distinct portions of particles of the first type of particles have an average particle size difference of at most 50 microns.

In some embodiments of the invention, the first type of particles is present in an amount of 2-60% by weight of the population of particles. In some embodiments of the invention, the first type of particles is present in an amount of 5-60% by weight of the population of particles. In some embodiments of the invention, the first type of particles is present in an amount of 10-60% by weight of the population of particles. In some embodiments of the invention, the first type of particles is present in an amount of 20-60% by weight of the population of particles. In some embodiments of the invention, the first type of particles is present in an amount of 30-60% by weight of the population of particles. In some embodiments of the invention, the first type of particles is present in an amount of 40-60% by weight of the population of particles. In some embodiments of the invention, the first type of particles is present in an amount of 2-50% by weight of the population of particles. In some embodiments of the invention, the first type of particles is present in an amount of 2-40% by weight of the population of particles. In some embodiments of the invention, the first type of particles is present in an amount of 2-30% by weight of the population of particles.

In some embodiments of the invention, the one or more active pharmaceutical ingredients comprises two or more active pharmaceutical ingredients.

In some embodiments of the invention, the one or more active pharmaceutical ingredients comprises three or more active pharmaceutical ingredients.

In some embodiments of the invention, the one or more active pharmaceutical ingredients comprises three or more active pharmaceutical ingredients comprises acetaminophen, phenyl ephrine, and dextromethorphan.

In some embodiments of the invention, the one or more active pharmaceutical ingredients comprises three or more active pharmaceutical ingredients comprises acetaminophen, dextromethorphan, and caffeine.

In some embodiments of the invention, the one or more active pharmaceutical ingredients comprises three or more active pharmaceutical ingredients comprises acetaminophen, dextromethorphan, and guaifenisine.

In some embodiments of the invention, the one or more active pharmaceutical ingredients comprises three or more active pharmaceutical ingredients comprises acetaminophen, phenylephrine, and guaifenisine.

In some embodiments of the invention, the one or more active pharmaceutical ingredients comprises three or more active pharmaceutical ingredients comprises acetaminophen, phenylephrine, guaifenisine, and dextromethorphan.

In some embodiments of the invention, the one or more active pharmaceutical ingredients comprises a combination of two or more low dose active pharmaceutical ingredients.

In some embodiments of the invention, the one or more active pharmaceutical ingredients comprises a combination of two or more medium dose active pharmaceutical ingredients.

In some embodiments of the invention, the one or more active pharmaceutical ingredients comprises a combination of two or more high dose active pharmaceutical ingredients.

In some embodiments of the invention, the one or more active pharmaceutical ingredients comprises a combination of one or more low and medium dose active pharmaceutical ingredients.

In some embodiments of the invention, the one or more active pharmaceutical ingredients comprises a combination of one or more low and high dose active pharmaceutical ingredients.

In some embodiments of the invention, the one or more active pharmaceutical ingredients comprises a combination of one or more low, medium, and high dose active pharmaceutical ingredients.

In some embodiments of the invention, the one or more pharmaceutical ingredients is microencapsulated.

In some embodiments of the invention, the one or more pharmaceutical ingredients is granulated with the carrier.

In some embodiments of the invention, the carrier for the one or more pharmaceutical ingredients comprises microcrystalline cellulose and starch, such as pregelatinized starch.

In some embodiments of the invention, the carrier for the one or more pharmaceutical ingredients comprises one or more sugar alcohols selected from the group consisting of erythritol, maltitol, xylitol, isomalt, lactitol, mannitol, sorbitol, and combinations thereof.

In some embodiments of the invention, the carrier for the one or more pharmaceutical ingredients comprises one or more binders from the group consisting of hydroxypropyl cellulose (HPC), hydroxypropylmethyl cellulose (HPMC), carboxymethyl cellulose (CMC), low substituted hydroxypropyl cellulose (L-HPC), polyvinylpyrrolidone (PVP), copovidone, and any mixture and combination thereof.

In some embodiments of the invention, further comprising a disintegrant.

In some embodiments of the invention, further comprising a disintegrant selected from the group consisting of sodium croscarmellose, crospovidone, sodium starch glycolate, and combinations thereof.

In some embodiments of the invention, further comprising one or more high-intensity sweeteners.

Preferred high intensity sweeteners include, but are not limited to sucralose, aspartame, salts of acesulfame, alitame, saccharin and its salts, cyclamic acid and its salts, glycyrrhizin, dihydrochalcones, thaumatin, monellin, stevioside (natural high intensity sweetener) and the like, alone or in combination. In order to provide longer lasting sweetness and flavor perception, it may be desirable to encapsulate or otherwise control the release of at least a portion of the artificial sweeteners. Techniques such as wet granulation, wax granulation, spray drying, spray chilling, fluid bed coating, conservation, encapsulation in yeast cells and fiber extrusion may be used to achieve desired release characteristics. Encapsulation of sweetening agents can also be provided using another component such as a resinous compound.

Usage level of the high intensity sweetener will vary considerably and will depend on factors such as potency of the sweetener, rate of release, desired sweetness of the product, level and type of flavor used and cost considerations. Thus, the active level of high intensity sweetener may vary from about 0.001 to about 8% by weight (preferably from about 0.02 to about 8% by weight). When carriers used for encapsulation are included, the usage level of the encapsulated sweetener will be proportionately higher. Combinations of sugar and/or non-sugar sweeteners may be used in the formulation.

In some embodiments of the invention, further comprising calcium carbonate or talc.

In some embodiments of the invention, further comprising a glidant.

In some embodiments of the invention, further comprising a glidant selected from the group consisting of colloidal silicon dioxide, starch, talc, tribasic calcium phosphate, magnesium stearate, magnesium carbonate, magnesium silicate, silica derivatives, and combinations thereof.

In some embodiments of the invention, further comprising a buffering agent.

In some embodiments of the invention, further comprising an effervescence system of a base and an acid.

In some embodiments of the invention, comprising an outer package material enclosing the population of particles.

In some embodiments of the invention, comprising an outer aluminum package material enclosing the population of particles

In some embodiments of the invention, comprising an outer oxygen impermeable package material enclosing the population of particles.

In some embodiments of the invention, the population of particles are administered directly in the mouth.

In some embodiments of the invention, the population of particles is poured into water and the water is administered in the mouth.

In some embodiments of the invention, the population of particles provides an improved mouthfeel compared to a powder mixture without the second type of particles, the improved mouthfeel including at least one of less sandy mouthfeel, less dusty mouthfeel, less roughness mouthfeel, less sticky or improved texture.

In some embodiments of the invention, the population of particles provides faster dissolution compared to a powder mixture without the second type of particles.

In some embodiments of the invention, the population of particles provides improved taste masking compared to a powder mixture without the second type of particles.

In some embodiments of the invention, the population of particles provides improved taste masking compared to an oral tablet comprising a combination of directly compressible (DC) sugar alcohol particles and non-directly compressible (non-DC).

In some embodiments of the invention, the population of particles provides improved taste masking compared to an oral tablet comprising a combination of directly compressible (DC) sugar alcohol particles and non-directly compressible (non-DC) in a ratio of 0.2 and 1.2, such as 0.3 to 0.7.

In some embodiments of the invention, the population of particles has a unit weight of 200 to 500 mg in the flow pack.

In some embodiments of the invention, the population of particles has a unit weight of 500 to 2000 mg in the flow pack.

In some embodiments of the invention, the population of particles has a unit weight of 700 to 2000 mg in the flow pack.

In some embodiments of the invention, the population of particles has a unit weight of 1000 to 2000 mg in the flow pack.

In another aspect of the invention there is provided a flowpack for oral delivery of active ingredients comprising a population of particles, an outer package material enclosing the population of particles, the population of particles comprising:
a first type of particles comprising one or more distinct portions of particles with a content of one or more active pharmaceutical ingredients and a carrier for the one or more active pharmaceutical ingredients;
a second type of particles in an amount of 30-95% by weight of the population of particles separate from the first type of particles, the second type of particles comprising at least 95% by weight of one or more sweeteners; and
a third type of particles in an amount of 1-10% by weight of the population of particles separate from the first and second type of particles, the third type of particles comprising flavor.

In another aspect of the invention there is provided a use of a flowpack as a powder delivery system for improving saliva generation.

In another aspect of the invention there is provided a flowpack as a powder delivery system for administration of active ingredients.

In another aspect of the invention there is provided a flowpack as a powder delivery system for improved taste masking of active pharmaceutical ingredients.

In another aspect of the invention there is provided a method of achieving improved taste masking of active pharmaceutical ingredients, comprising the steps of:
a) providing a flow pack comprising a population of particles according to the invention, the population of particles being a dry and flowable population of particles contained in an outer flowpack material,
b) resembling a liquid mouthwash by swishing said population of particles, thereby generating fluid in the oral cavity without adding water.

In the present context, “fluid” or “fluid generation” or similar wording is to be understood in context with the invention as “saliva” or “saliva generation” as a result of the administration of the powder delivery system according to the invention. Hence, standard saliva generation is not the intended meaning, but excess saliva generation directly triggered by the powder delivery system is part of the context. When the population of particles is mentioned to resemble a liquid mouthwash, the intended meaning is that enough liquid is generated to attribute the same or improved benefits as in liquid mouthwashes. The same amount of liquid as used in a liquid mouthwash is not needed according to the invention, just as long as the amount of saliva generated would be manageable to be “swished”. In some instances, however, the amount of saliva generated may be on the same level as by using a liquid mouthwash.

In another aspect of the invention there is provided a method of achieving improved salivation, comprising the steps of:
a) providing a flow pack comprising a population of particles according to the invention, the population of particles being a dry and flowable population of particles contained in an outer flowpack material,
b) resembling a liquid mouthwash by swishing said population of particles, thereby generating fluid in the oral cavity without adding water.

In another aspect of the invention there is provided a method of achieving improved salivation, comprising the steps of:
a) providing a flow pack comprising a population of particles according to the invention, the population of particles being a dry and flowable population of particles contained in an outer flowpack material,
b) subjecting the population of particles into water, thereby obtaining an at least partly dissolved swishable powder delivery system, and
c) swishing the at least partly dissolved swishable powder delivery system, thereby generating fluid in the oral cavity.

The meaning of “swishable” in the present context is to be understood as forcing either the powder delivery system around in the oral cavity or the liquid generated after a short period of time around in the oral cavity. The term “swishable” can also cover “gargling”, “swirling” or “pushing around” or similar expressions. The idea is that the powder delivery system and the fluid generated is to be distributed in the oral cavity, both to oral mucosa, tongue and teeth in a way that it secures contact to the surfaces in the oral cavity. Upon “swishing”, the idea is also that the portion of liquid generated is not to be swallowed during the operation, although a low amount is allowed to be swallowed while maintaining a major part of the saliva generated in the oral cavity in order to resemble a liquid mouthwash. Usually, the system is strong enough to avoid adding additional water. However, water may be added to some extent but would not be required in order to deliver the advantages of the present invention.

In some embodiments of the invention, swishing said powder delivery system is characterised by forcing the powder delivery system around the oral cavity for a period of time.

In some embodiments of the invention, swishing said powder delivery system is characterised by forcing the powder delivery system around the oral cavity for at least 5 seconds. In some embodiments of the invention, swishing said powder delivery system is characterised by forcing the powder delivery system around the oral cavity for at least 10 seconds. In some embodiments of the invention, swishing said powder delivery system is characterised by forcing the powder delivery system around the oral cavity for at least 15 seconds. In some embodiments of the invention, swishing said powder delivery system is characterised by forcing the powder delivery system around the oral cavity for at least 20 seconds.

In some embodiments of the invention, at least a portion of the fluid generated by swishing said powder delivery system is forced around the oral cavity for a period of time.

Surprisingly, the powder delivery system in some embodiments was able to dissolve relatively quickly after oral administration in the oral fluid generated upon administration. In some embodiments, the powder delivery system dissolve within 15 seconds. In some embodiments, the powder delivery system dissolve within 10 seconds. In some embodiments, the powder delivery system dissolve within 8 seconds. In some embodiments, the powder delivery system dissolve within 5 seconds.

In some embodiments of the invention, at least a portion of the saliva generated by swishing said powder delivery system is forced around the oral cavity for at least 10 seconds. In some embodiments of the invention, at least a portion of the saliva generated by swishing said powder delivery system is forced around the oral cavity for at least 20 seconds. In some embodiments of the invention, at least a portion of the saliva generated by swishing said powder delivery system is forced around the oral cavity for at least 30 seconds.

In some embodiments of the invention, at least a portion of the fluid generated by swishing said powder delivery system is forced around the oral cavity for a period of time prior to swallowing or spitting out said portion of fluid.

In some embodiments of the invention, benefits are obtained by swishing said powder delivery system and/or at least a portion of the fluid generated in the oral cavity for at least 10 seconds. In some embodiments of the invention, benefits are obtained by swishing said powder delivery system and/or at least a portion of the fluid generated in the oral cavity for at least 10 seconds for at least 20 seconds. In some embodiments of the invention, benefits are obtained by swishing said powder delivery system and/or at least a portion of the fluid generated in the oral cavity for at least 30 seconds.

In some embodiments of the invention, the powder delivery system is a dry and flowable population of particles that is swished upon oral administration and generates fluid in the oral cavity, optionally adding water, and thereby resembling a liquid mouthwash.

In some embodiments of the invention, the powder delivery system generates more than 1.5 mL fluid in the oral cavity within a period from 30 to 90 seconds from onset of administration.

In some embodiments of the invention, the powder delivery system generates more than 1.5 mL fluid in the oral cavity within a period from 90 to 180 seconds from onset of administration.

In some embodiments of the invention, the powder delivery system generates more than 1.5 mL fluid in the oral cavity within a period from 180 to 300 seconds from onset of administration.

### DETAILED DESCRIPTION

The verb “to comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”. Additionally, the words “a” and “an” when used in the present document in connection with the word comprising or containing denote “one or more.” The expression “one or more” is intended to mean one, two, three or more.

As used herein, the term “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein, the term “%” and “percent” refers to percent by weight, unless otherwise is stated.

In the present context, the phrase “population of particles” refers to a statistical population of particles. The population of particles may be characterized by a number of different parameters, e.g. statistical parameters such as distribution of particles, average particle size, particle size distribution width, etc. The population of particles may have subpopulations.

The term “particle size” relates to the ability of the particles to move through or be retained by sieve holes of a specific size. As used herein, the term “particle size” refers to the average particle size as determined according to European Pharmacopoeia 9.1 when using test method 2.9.38 particle size distribution estimation by analytical sieving, unless otherwise specifically is mentioned.

The term “particle” or similar wording is intended to denote a single, discrete composition of solid matter, such as a granule or individual elements in powder, having a certain size that may deviate considerable.

The term “flow pack” is intended to mean a wrapping containing the powder system according to the present invention, where the package is generally given the meaning in the field of flowpack technology. Generally, the powder system is applied during “flow” of the wrapping material in a machinery that allows an efficient process with high speed. Stick packs and sachets are examples of flow packs.

The term “powder system”, “powder delivery system” or “population of particles” is intended to be understood as the entire content of matter filled into the flowpack according to the invention, i.e. excluding the package or wrapping material surrounding the content.

As used herein, the term “dissolve” is the process where a particle enters a solvent (oral saliva) to yield a solution. Unless otherwise stated, dissolving implies a full dissolving of the compound in question. In some embodiments, the dissolution rate of the active ingredient is measured and shows an improvement compared to conventional powder formulations.

The term “in vivo release” or “in vivo testing of release” or similar wording intends to mean that the formulation is tested as outlined in the examples.

The term “in vitro release” or “in vitro testing of release” or similar wording intends to mean that the formulation is tested according to the examples, in particular according to General Monograph 2.9.25 in European Pharmacopoeia, 5th ed.

The term “release” in the present context is intended to mean under “in vitro” conditions if not stated otherwise. In particular, the “release rate” during a certain period of time is intended to mean the amount in percentage of active ingredient that is released during the period. In some embodiments, the process of releasing a substance corresponds to the substance being dissolved in saliva.

The term “sustained release” or “extended release” is herein intended to mean prolonged release over time. The term “rapid release” or “quick release” or “high release” is herein intended to mean a higher content released for a given period of time.

When referring to “initial watering effect” (increased saliva generation), “mouthfeel”, “taste masking”, and “liking score”, the tests were made using the following method, unless stated otherwise. Test subjects abstain from eating and drinking at least 30 minutes before initiation of any test. Immediately before introducing of the powder sample or tablet into the oral cavity, the test subject swallows. The test subject refrains from swallowing during the test. Immediately after introducing of the tablet into the oral cavity, the test subject starts masticating the tablet at a frequency of 1 chew per second for 20 seconds. In case of powder samples, the test subject moves the sample around using the tongue at a frequency of 1 back and forth per second for 20 seconds. Then, saliva and any remains of the powder sample or tablet is kept in the mouth for 10 seconds. During this period, the test subject evaluates the sensorial parameters “mouth feel”, “taste masking” and “liking”. 30 seconds after starting the test, the test subject discards saliva including any powder or tablet fragments into a plastic cup, which is weighted. Saliva discarded also at 90 seconds after onset of mastication, at 180 seconds after onset of mastication, at 300 seconds after onset of mastication, at 420 seconds after onset of mastication, and at 600 seconds after onset of mastication. At all times, the test subject makes as little movement as possible, and refrains from swallowing.

The term “delivery to the oral mucosa” or similar wording intends to mean that the formulation is tested according to the examples.

A “self-emulsifying agent” is an agent which will form an emulsion when presented with an alternate phase with a minimum energy requirement. In contrast, an emulsifying agent, as opposed to a self-emulsifying agent, is one requiring additional energy to form an emulsion.

Due to the poor solubility of certain active ingredients in physiological fluids, it is an unmet need to have a high dose of certain active ingredients in a form, that solubilize the active ingredient upon mixture with the body physiological fluids to facilitate bio-absorption. To overcome low oral bioavailability, various lipid-based drug delivery systems and self-emulsifying systems have been developed. Lipid-based delivery systems and particularly self-emulsifying drug delivery systems (SEDDS) have been demonstrated to increase the solubility, dissolution, and bioavailability of many insoluble active ingredients. However, lipid-based and SEDDS delivery systems are very limited by the amount of active ingredient loading that has to be dissolved in the vehicle composition. Higher concentration of active ingredients is obtained using co-solvents, which enable loads of up to 30% in specific cases.

Particular challenges are considered to arise in formulating powders with SEDDS. For instance, challenges may arise with obtaining a homogenous mixture where variations are avoided and a safe and convenient delivery may be obtained. Also, the general formulation of the oral powders offering convenience to the user need not be compromised which is often the case if precaution is not taken, such as in cases where a high load of active ingredients is needed.

Particularly with respect to SEDDS, the formulation of the present invention may provide some clear benefits, both allowing a higher load of active ingredients and at the same time offer improved sensorics properties of the formulation during use. Other advantages are also present.

Importantly, the presence of SEDDS or at least a self-emulsifying agent was seen to act in synergy with increased saliva generation. While increased saliva generation was seen to distribute certain active ingredients and allocate a higher load of active ingredients to for instance mucosal surfaces, the presence of SEDDS or at least a self-emulsifying agent was seen to further increase the uptake of these active ingredients through oral surfaces. Accordingly, the synergy between the presence of SEDDS or at least a self-emulsifying agent and increased saliva generation according to the invention was a surprise to the inventors. In some embodiments, increased saliva generation may result in a higher exposure of the active ingredients to mucosal surfaces. The presence of SEDDS may work to increase the affinity of the active ingredients from this saliva to the mucosa. Particularly, the potential of SEDDS to have a high load of active ingredients further contributes to the synergy of the powder according to the invention in combination with improved saliva generation.

In the present context, SEDDS is a solid or liquid dosage form comprising an oil phase, a surfactant and optionally a co-surfactant, characterized primarily in that said dosage form can form oil-in-water emulsion spontaneously in the oral cavity or at ambient temperature (referring generally to body temperature, namely 37° C.). When a SEDDS enters the oral cavity, it is initially self-emulsified as emulsion droplets and rapidly dispersed throughout the oral cavity, and thus reducing the irritation caused by the direct contact of the active ingredient with the mucous membrane of the oral cavity, and hence helping on taste-masking active ingredients. In the oral cavity, the structure of the emulsion microparticulate will be changed or destroyed. The resulting microparticulate of micrometer or nanometer level can penetrate into the mucous membrane of for instance the oral cavity, and the absorbed oil droplets enter the blood circulation, thereby significantly improving the bioavailability of the active ingredient.

In an embodiment of the invention, the self-emulsifying system comprises one or more emulsifiers and one or more oil carriers.

In an embodiment of the invention, the self-emulsifying system comprises one or more emulsifiers, one or more oil carriers and one or more solubilizers.

In an embodiment of the invention, the self-emulsifying system comprises one or more emulsifiers, one or more oil carriers, one or more solubilizers and one or more solvents.

In an embodiment of the invention, the self-emulsifying system comprises one or more emulsifiers and one or more solvents.

In an embodiment of the invention, the self-emulsifying system comprises one or more emulsifiers that have both emulsifying and solubilizing properties.

In an embodiment of the invention, the self-emulsifying system comprises one or more emulsifiers that act as both an emulsifier and a carrier.

In an embodiment of the invention, the self-emulsifying system comprises one or more emulsifiers that act as both an emulsifier, a carrier and a solubilizer.

In an embodiment of the invention, the self-emulsifying system comprises one or more fatty acids, one or more glycerols, one or more waxes, one or more flavonoids and one or more terpenes.

In an embodiment of the invention, the self-emulsifying system comprises one or more emulsifiers that have an HLB-value of more than 6, preferably of 8-18.

In an embodiment of the invention, the one or more emulsifiers are selected from the group consisting of PEG-35 castor oil, PEG-6 oleoyl glycerides, PEG-6 linoleoyl glycerides, PEG-8 caprylic/capric glyceride, sorbitan monolaurate, sorbitan monooleate, polyoxyethylene (20) sorbitan monolaurate, polyoxyethylene (60) sorbitan monostearate, polyoxyethylene (80) sorbitan monooleate, lauroylpoloxyl-32 glycerides, stearoyl polyoxyl-32 glycerides, polyoxyl-32 stearate, propylene glycol mono laurate, propylene glycol di laurate, and mixtures and combinations thereof.

In an embodiment of the invention, the one or more emulsifiers comprise PEG-35 castor oil.

In an embodiment of the invention, the oil carrier is selected from the group consisting of natural fatty acids; medium-chain triglycerides of caprylic (C8) and capric (C10) acids; propylene glycol esters of caprylic (C8) and capric (C10) acids; mono-, di- and triglycerides of mainly linoleic (C18:2) and oleic (C18:1) acids; fatty acid 18:1 cis-9; natural fatty acids; mono-, di- and triglycerides of oleic (C18:1) acid, and mixtures and combinations thereof.

In an embodiment of the invention, the one or more solvents are selected from the group consisting of polyglyceryl-3 dioleate, 1,2-propandiol, polyethylene glycol 300, polyethylene glycol 400, diethylene glycol monoethyl ether, and mixtures and combinations thereof.

In an embodiment of the invention, the oil carrier is selected from the group consisting of corn oil, Labrafac lipophile WL1349, Labrafac PG, Maisine CC, oleic acid, olive oil, Peceol, and mixtures and combinations thereof.

In an embodiment of the invention, the one or more solvents are selected from the group consisting of polyglyceryl-3 dioleate, 1,2-propandiol, polyethylene glycol 300, polyethylene glycol 400, diethylene glycol monoethyl ether, and mixtures and combinations thereof.

In an embodiment of the invention, the one or more solubilizers are selected from the group consisting of lauroylpoloxyl-32 glycerides; stearoyl polyoxyl-32 glycerides; Polyoxyl-32 stearate; synthetic copolymer of ethylene oxide (80) and propylene oxide (27); polyvinyl caprolactam-polyvinyl acetate-polyethylene glycol graft co-polymer; alpha-, beta- or gamma cyclodextrins and derivatives thereof; pea proteins (globulins, albumins, glutelins proteins); and mixtures and combinations thereof.

In an embodiment of the invention, the powder comprises an active pharmaceutical ingredient and a self-emulsifying system that when hydrated with saliva upon oral administration forms an emulsion.

In an embodiment of the invention, the oral powders further comprise one or more lipids.

Antioxidants suitable for use include butylated hydroxyanisole (BHA), butylated hydroxytoluene (BHT), betacarotenes, tocopherols, acidulants such as Vitamin C (ascorbic acid or corresponding salts (ascorbates)), propyl gallate, catechins, green tea extract other synthetic and natural types or mixtures thereof.

High intensity sweetening agents can also be used according to preferred embodiments of the invention. Preferred high intensity sweeteners include, but are not limited to sucralose, aspartame, salts of acesulfame, alitame, neotame, saccharin and its salts, cyclamic acid and its salts, glycyrrhizin, dihydrochalcones, thaumatin, monellin, monk fruit extract, advantame, stevioside and the like, alone or in combination.

In order to provide longer lasting sweetness and flavor perception, it may be desirable to encapsulate or otherwise control the release of at least a portion of the high intensity sweeteners.

Techniques such as wet granulation, wax granulation, spray drying, spray chilling, fluid bed coating, conservation, encapsulation in yeast cells and fiber extrusion may be used to achieve desired release characteristics. Encapsulation of sweetening agents can also be provided using another formulation component such as a resinous compound.

Usage level of the high-intensity sweetener will vary considerably and will depend on factors such as potency of the sweetener, rate of release, desired sweetness of the product, level and type of flavor used and cost considerations. Thus, the active level of artificial sweetener may vary from about 0.001 to about 8% by weight (preferably from about 0.02 to about 8% by weight). When carriers used for encapsulation are included, the usage level of the encapsulated high-intensity sweetener will be proportionately higher.

In one embodiment examples of active substances includes a comprehensive list of which is found e.g. in WO 00/25598, which is incorporated herein by reference, including drugs, dietary supplements, antiseptic agents, pH adjusting agents, anti-smoking agents and substances for the care or treatment of the oral cavity and the teeth such as hydrogen peroxide and compounds capable of releasing urea during chewing. Examples of useful active substances in the form of antiseptics include salts and derivatives of guanidine and biguanidine (for instance chlorhexidine diacetate) and the following types of substances with limited water-solubility: quaternary ammonium compounds (e.g. ceramine, chloroxylenol, crystal violet, chloramine), aldehydes (e.g. paraformaldehyde), derivatives of dequaline, polynoxyline, phenols (e.g. thymol, p-chlorophenol, cresol), hexachlorophene, salicylic anilide compounds, triclosan, halogenes (iodine, iodophores, chloroamine, dichlorocyanuric acid salts), alcohols (3,4 dichlorobenzyl alcohol, benzyl alcohol, phenoxyethanol, phenylethanol), cf. also Martindale, The Extra Pharmacopoeia, 28th edition, pages 547-578; metal salts, complexes and compounds with limited water-solubility, such as aluminum salts, (for instance aluminum potassium sulphate AlK(SO4)2, 12H2O) and salts, complexes and compounds of boron, barium, strontium, iron, calcium, zinc, (zinc acetate, zinc chloride, zinc gluconate), copper (copper chloride, copper sulphate), lead, silver, magnesium, sodium, potassium, lithium, molybdenum, vanadium should be included; other compositions for the care of mouth and teeth: for instance; salts, complexes and compounds containing fluorine (such as sodium fluoride, sodium monofluorophosphate, aminofluorides, stannous fluoride), phosphates, carbonates and selenium. Further active substances can be found in J. Dent. Res. Vol. 28 No. 2, pages 160-171, 1949.

Examples of active substances in the form of agents adjusting the pH in the oral cavity include: acids, such as adipic acid, succinic acid, fumaric acid, or salts thereof or salts of citric acid, tartaric acid, malic acid, acetic acid, lactic acid, phosphoric acid and glutaric acid and acceptable bases, such as carbonates, hydrogen carbonates, phosphates, sulphates or oxides of sodium, potassium, ammonium, magnesium or calcium, especially magnesium and calcium.

Active ingredients may comprise the below mentioned compounds or derivates thereof but are not limited thereto: Acetaminophen, Acetylsalicylic acid, Buprenorphine, Bromhexin, Celcoxib, Codeine, Diphenhydramineclofenac, Etoricoxib, Ibuprofen, Indometacin, Ketoprofen, Lumiracoxib, Morphine, Naproxen, Oxycodon, Parecoxib, Piroxicam, Pseudoefedrin, Rofecoxib, Tenoxicam, Tramadol, Valdecoxib, Calciumcarbonat, Magaldrate, Disulfiram, Bupropion, Nicotine, Azithromycin, Clarithromycin, Clotrimazole, Erythromycin, Tetracycline, Granisetron, Ondansetrone, Prometazin, Tropisetron, Brompheniramine, Ceterizin, leco-Ceterizin, Chlorcyclizine, Chlorpheniramin, Chlorpheniramin, Difenhydramine, Doxylamine, Fenofenadin, Guaifenesin, Loratidin, des-Loratidin, Phenyltoloxamine, Promethazin, Pyridamine, Terfenadin, Troxerutin, Methyldopa, Methylphenidate, Benzalcon. Chloride, Benzeth. Chloride, Cetylpyrid. Chloride, Chlorhexidine, Ecabet-sodium, Haloperidol, Allopurinol, Colchinine, Theophylline, Propanolol, Prednisolone, Prednisone, Fluoride, Urea, Actot, Glibenclamide, Glipizide, Metformin, Miglitol, Repaglinide, Rosiglitazone, Apomorfin, Cialis, Sildenafil, Vardenafil, Diphenoxylate, Simethicone, Cimetidine, Famotidine, Ranitidine, Ratinidine, cetrizin, Loratadine, Aspirin, Benzocaine, Dextrometorphan, Phenylpropanolamine, Pseudoephedrine, Cisapride, Domperidone, Metoclopramide, Acyclovir, Dioctylsulfosucc, Phenolphtalein, Almotriptan, Eletriptan, Ergotamine, Migea, Naratriptan, Rizatriptan, Sumatriptan, Zolmitriptan, Aluminum salts, Calcium salts, Ferro salts, Ag-salts, Zinc-salts, Amphotericin B, Chlorhexidine, Miconazole, Triamcinolonacetonid, Melatonine, Phenobarbital, Caffeine, Benzodiazepiner, Hydroxyzine, Meprobamate, Phenothiazine, Buclizine, Brometazine, Cinnarizine, Cyclizine, Difenhydramine, Dimenhydrinate, Buflomedil, Amphetamine, Caffeine, Ephedrine, Orlistat, Phenylephedrine, Phenylpropanolamin, Pseudoephedrine, Sibutramin, Ketoconazole, Nitroglycerin, Nystatin, Progesterone, Testosterone, Vitamin B12, Vitamin C, Vitamin A, Vitamin D, Vitamin E, Pilocarpin, Aluminumaminoacetat, Cimetidine, Esomeprazole, Famotidine, Lansoprazole, Magnesiumoxide, Nizatide and or Ratinidine.

The invention is suitable for increased or accelerated release of active agents selected among the group of dietary supplements, oral and dental compositions, antiseptic agents, pH adjusting agents, anti-smoking agents, sweeteners, flavorings, aroma agents or drugs. Some of those will be described below.

The active agents to be used in connection with the present invention may be any substance desired to be released from the powder. The active agents, for which a controlled and/or accelerated rate of release is desired, are primarily substances with a limited water-solubility, typically below 10 g/100 mL inclusive of substances which are totally water-insoluble. Examples are medicines, dietary supplements, oral compositions, anti-smoking agents, highly potent sweeteners, pH adjusting agents, flavorings etc.

Other active ingredients are, for instance, paracetamol, benzocaine, cinnarizine, menthol, carvone, caffeine, chlorhexidine-di-acetate, cyclizine hydrochloride, 1,8-cineol, nandrolone, miconazole, mystatine, sodium fluoride, nicotine, cetylpyridinium chloride, other quaternary ammonium compounds, vitamin E, vitamin A, vitamin D, glibenclamide or derivatives thereof, progesterone, acetylsalicylic acid, dimenhydrinate, cyclizine, metronidazole, sodium hydrogen carbonate, the active components from Ginkgo, the active components from propolis, the active components from ginseng, methadone, oil of peppermint, salicylamide, hydrocortisone or astemizole.

Examples of active agents in the form of dietary supplements are for instance salts and compounds having the nutritive effect of vitamin B2 (riboflavin), B12, folinic acid, folic acid, niacine, biotine, poorly soluble glycerophosphates, amino acids, the vitamins A, D, E and K, minerals in the form of salts, complexes and compounds containing calcium, phosphorus, magnesium, iron, zinc, copper, iodine, manganese, chromium, selenium, molybdenum, potassium, sodium or cobalt.

Furthermore, reference is made to lists of nutritionists accepted by the authorities in different countries such as for instance US code of Federal Regulations, Title 21, Section 182.5013.182 5997 and 182.8013-182.8997.

Examples of active agents in the form of antiseptics are for instance salts and compounds of guanidine and biguanidine (for instance chlorhexidine diacetate) and the following types of substances with limited water-solubility: quaternary ammonium compounds (for instance ceramine, chloroxylenol, crystal violet, chloramine), aldehydes (for instance paraformaldehyde), compounds of dequaline, polynoxyline, phenols (for instance thymol, para chlorophenol, cresol) hexachlorophene, salicylic anilide compounds, triclosan, halogenes (iodine, iodophores, chloroamine, dichlorocyanuric acid salts), alcohols (3,4 dichlorobenzyl alcohol, benzyl alcohol, phenoxyethanol, phenylethanol), cf. furthermore Martindale, The Extra Pharmacopoeia, 28th edition, pages 547-578; metal salts, complexes and compounds with limited water-solubility, such as aluminum salts, (for instance aluminum potassium sulphate AlK(SO4)2, 12H2O) and furthermore salts, complexes and compounds of boron, barium, strontium, iron, calcium, zinc, (zinc acetate, zinc chloride, zinc gluconate), copper (copper chloride, copper sulfate), lead, silver, magnesium, sodium, potassium, lithium, molybdenum, vanadium should be included; other compositions for the care of mouth and teeth: for instance; salts, complexes and compounds containing fluorine (such as sodium fluoride, sodiummonofluorophosphate, amino fluorides, stannous fluoride), phosphates, carbonates and selenium.

Cf. furthermore J. Dent. Res. Vol. 28 No. 2, pages 160-171, 1949, wherein a wide range of tested compounds is mentioned.

Examples of active agents in the form of agents adjusting the pH in the oral cavity include for instance: acceptable acids, such as adipic acid, succinic acid, fumaric acid, or salts thereof or salts of citric acid, tartaric acid, malic acid, acetic acid, lactic acid, phosphoric acid and glutaric acid and acceptable bases, such as carbonates, hydrogen carbonates, phosphates, sulfates or oxides of sodium, potassium, ammonium, magnesium or calcium, especially magnesium and calcium.

Examples of active agents in the form of anti-smoking agents include for instance: nicotine, tobacco powder or silver salts, for instance silver acetate, silver carbonate and silver nitrate.

Further examples of active agents are medicines of any type.

Examples of active agents in the form of medicines include caffeine, salicylic acid, salicyl amide and related substances (acetylsalicylic acid, choline salicylate, magnesium salicylate, sodium salicylate), paracetamol, salts of pentazocine (pentazocine hydrochloride and pentazocinelactate), buprenorphine hydrochloride, codeine hydrochloride and codeine phosphate, morphine and morphine salts (hydrochloride, sulfate, tartrate), methadone hydrochloride, ketobemidone and salts of ketobemidone (hydrochloride), beta-blockers, (propranolol), calcium antagonists, verapamil hydrochloride, nifedinpine as well as suitable substances and salts thereof mentioned in Pharm. Int., November 85, pages 267-271, Barney H. Hunter and Robert L. Talbert, nitroglycerine, erythrityl tetranitrate, strychnine and salts thereof, lidocaine, tetracaine hydrochloride, etorphine hydrochloride, atropine, insulin, enzymes (for instance papain, trypsin, amyloglucosidase, glucoseoxidase, streptokinase, streptodornase, dextranase, alpha amylase), polypeptides (oxytocin, gonadorelin, (LH.RH), desmopressin acetate (DDAVP), isoxsuprine hydrochloride, ergotamine compounds, chloroquine (phosphate, sulfate), isosorbide, demoxytocin, heparin.

Other active ingredients include beta-lupeol, Letigen®, Sildenafil citrate and derivatives thereof.

Further examples of active ingredients include dental products including Carbamide, CPP Caseine Phospho Peptide; Chlorhexidine, Chlorhexidine di acetate, Chlorhexidine Chloride, Chlorhexidine di gluconate, Hexetedine, Strontium chloride, Potassium Chloride, Sodium bicarbonate, Sodium carbonate, Fluor containing ingredients, Fluorides, Sodium fluoride, Aluminum fluoride.

Further examples of active ingredients include Ammonium fluoride, Calcium fluoride, Stannous fluoride, Other fluor containing ingredients Ammonium fluorosilicate, Potassium fluorosilicate, Sodium fluorosilicate, Ammonium monofluorophosphate, Calcium monofluorophosphate, Potassium monofluorophosphate, Sodium monofluorophosphate, Octadecenyl Ammonium fluoride, Stearyl Trihydroxyethyl Propylenediamine Dihydrofluoride

Further examples of active ingredients include vitamins. Vitamins include A, B1, B2, B6, B12, Folinic acid, Folic acid, niacin, Pantothenic acid, biotine, C, D, E, K. Minerals include Calcium, phosphor, magnesium, iron, Zinc, Copper, Iod, Mangan, Crom, Selene, Molybden. Other active ingredients include:

Q10®, enzymes. Natural drugs including Ginkgo biloba, ginger, and fish oil.

Further examples of active ingredients include migraine drugs such as Serotonin antagonists: Sumatriptan, Zolmitriptan, Naratriptan, Rizatriptan, Eletriptan; nausea drugs such as Cyclizin, Cinnarizin, Dimenhydramin, Difenhydrinat; hay fever drugs such as Cetrizin, Loratidin, pain relief drugs such as Buprenorfin, Tramadol, oral disease drugs such as Miconazol, Amphotericin B, Triamcinolonaceton; and the drugs Cisaprid, Domperidon, Metoclopramid. In a preferred embodiment the invention relates to the release of Nicotine and its salts.

In an advantageous embodiment of the invention the active ingredient is selected from active ingredients for the throat selected from acetylcysteine, ambroxol, amylmetacresol, benzocaine, bisacodyl, bismuth subsalicylate, bromhexine, cetirizine, cetylpyridinium, chlorhexidine, dextromethorphan hydrobromide, 2,4-dichlorobenzyl alcohol, doxylamine succinate, eucalyptus oil, flurbiprofen, glycerin, hexylresorcinol, lidocaine, menthol, myrrh, paracetamol, pectin, peppermint oil, phenol, phenylephrine, povidone-iodine, pseudoephedrine, ranitidine, simethicone, sodium docusate, spearmint, zinc, or any combination thereof; active ingredients for the gastrointestinal tract selected from alginate, atenolol, aspirin (acetylsalicylic acid), ampicillin, aminosalicylates, anhydrous citric acid, aspirin, bisacodyl, bismuth subsalicylate, bupropion, caffeine, calcium, calcium carbonate, cetirizine, cimetidine, cisapride, clarithromycin, desloratadine, dexlansoprazole, diphenhydramine HCl, diphenhydramine citrate, dimenhydrinate, docusate erythromycin, dopamine, esomeprazole, famotidine, fexofenadine HCl, guaifenesin, hydrotalcite, ibuprofen, ketoprofen, lactase enzyme, lansoprazole, loratadine, lorcaserin, loperamide, loperamide HCl, magnesium, magnesium carbonate, magnesium hydroxide, melatonin, methamphetamine HCl, metoclopramide, metronidazole, montelukast, mycostatin, naltrexone, naproxen, naproxen sodium, nizatidine, omeprazole, ondansetron, orlistat, pantoprazole, paracetamol (acetaminophen), pectin, phentermine HCl, polypodium leucotomos, prednisolone, prednisone, progesterone, propranolol, propantheline bromide, pseudoephedrine HCl, phentermine, rabeprazole, ranitidine, roflumilast, scopoloamine butyl hydroxide, simethicone, sodium, sodium bicarbonate, sodium docusate, sumatriptan, testosterone, tetracycline, topiramate, vitamin A, vitamin B, vitamin B12, vitamin C (ascorbic acid), vitamin D, and vitamin E, vitamin K, or any combination thereof, and active ingredients for buccal absorption selected from atenolol, baclofen, caffeine, carvedilol, chlorpheniramine, chlorpheniramine maleate, fluticasone propionate, maleate, desmopressin, diltiazem hydrochloride, doxylamine succinate, mycostatin, nicotine, nifedipine, nitroglycerin, omeprazole, ondansetron, oxymetazoline HCl, oxytocin, phenylephrine, piroxicam, prednisone, propranolol, salbutamol sulphate, scopoloamine butyl hydroxide, sumatriptan, triamcinolonacetonid, and any combination thereof.

A powder according to the invention may, if desired, include one or more fillers/texturisers including as examples, magnesium and calcium carbonate, sodium sulphate, ground limestone, silicate compounds such as magnesium and aluminum silicate, kaolin and clay, aluminum oxide, silicium oxide, talc, titanium oxide, mono-, di- and tri-calcium phosphates, cellulose polymers and combinations thereof.

In an embodiment of the invention, the formulation comprises further ingredients selected from the group consisting of flavors, dry-binders, anti-caking agents, emulsifiers, antioxidants, enhancers, mucoadhesives, absorption enhancers, high intensity sweeteners, softeners, colors, active ingredients, water-soluble indigestible polysaccharides, water-insoluble polysaccharides, or any combination thereof.

According to embodiments of the invention, the emulsifiers may be selected from the group consisting of sucrose ester of fatty acids (such as sucrose mono stearate), polyethylene glycol esters or ethers (PEG) (such as caprylocaproyl macrogol-8 glycerides and lauroyl macrogol-32-glycerides), mono- and diglyceride of fatty acids (such as glycerol monostearate, glycerol monolaurate, glyceryl behenate ester), acetic acid esters of mono- and diglycerides of fatty acids (Acetem), polyoxyethylene alkyl ethers, diacetyl tartaric ester of monoglycerides, lactylated monoglycerides, glycerophospholipids (such as lecithin), poloxamer (non-ionic block copolymer of ethylene oxide and propylene oxide), cyclodextrins, fatty acid esters of sorbitol (such as sorbitan monolaurate, sorbitan monostearate, sorbitan tristearate, polysorbates).

According to embodiments of the invention, flavors may be selected from the group consisting of coconut, coffee, chocolate, vanilla, grapefruit, orange, lime, menthol, liquorice, caramel aroma, honey aroma, peanut, walnut, cashew, hazelnut, almonds, pineapple, strawberry, raspberry, tropical fruits, cherries, cinnamon, peppermint, wintergreen, spearmint, eucalyptus, and mint, fruit essence such as from apple, pear, peach, strawberry, apricot, raspberry, cherry, pineapple, and plum essence. The essential oils include peppermint, spearmint, menthol, eucalyptus, clove oil, bay oil, anise, thyme, cedar leaf oil, nutmeg, and oils of the fruits mentioned above.

The active ingredient may also be one or more cannabinoids selected from: cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCV A). More preferably the one or more cannabinoid is CBD or THC.

In an embodiment of the invention, the formulation comprises particles comprising gum base, and wherein the formulation is designed to be masticated into a coherent residual containing water-insoluble components.

The application of gum may in the present context may invoke a delay of release for active ingredients and this may again promote the buccal and upper throat absorption of active pharmaceutical ingredient when this is released from the formulation during mastication.

In an embodiment of the invention, the formulation contains particles comprising gum base, and wherein the gum base comprises at least 5% by weight of elastomer.

### EXAMPLES

### Preparation of a Population of Particles

TABLE 1

| Raw material (wt %) | Ex1 | Ex2 | Ex3 | Ex4 | Ex5 | Ex6 |
|---|---|---|---|---|---|---|
| DC Xylitol | 50 | — | — | — | — | — |
| Sorbitol | — | 50 | — | — | — | — |
| DC Isomalt | — | — | 50 | — | — | — |
| DC Erythritol | — | — | — | 50 | — | — |
| DC Mannitol | — | — | — | — | 50 | — |
| DC Maltitol | — | — | — | — | — | 50 |
| Acetaminophen** | 46 | 46 | 46 | 46 | 46 | 46 |
| Flavor* | 4 | 4 | 4 | 4 | 4 | 4 |

*powder flavor.
**Including a granulation of 50% acetaminophen, 23% microcrystalline cellulose (MCC), 22% sorbitol and 5% starch (by weight).

TABLE 2

| Raw material (wt %) | Ex1A | Ex1B | Ex1C | Ex1D | Ex1E | Ex1F |
|---|---|---|---|---|---|---|
| DC Xylitol | 81 | 86 | 76 | 66 | 56 | 36 |
| Acetaminophen** | 5 | 10 | 20 | 30 | 40 | 60 |
| Flavor* | 4 | 4 | 4 | 4 | 4 | 4 |

*powder flavor.
**Including a granulation of 50% acetaminophen, 23% microcrystalline cellulose (MCC), 22% sorbitol and 5% starch (by weight).

TABLE 3

| Raw material (wt %) | Ex1G | Ex1H | Ex1I | Ex1J | Ex1K | Ex1L |
|---|---|---|---|---|---|---|
| DC Xylitol | 39 | 38 | 36 | 35 | 32 | 30 |
| Acetaminophen** | 60 | 60 | 60 | 60 | 60 | 60 |
| Flavor* | 1 | 2 | 4 | 5 | 8 | 10 |

*powder flavor.
**Including a granulation of 50% acetaminophen, 23% microcrystalline cellulose (MCC), 22% sorbitol and 5% starch (by weight).

TABLE 4

| Raw material (wt %) | Ex7 | Ex8 | Ex9 | Ex10 | Ex11 | Ex12 |
|---|---|---|---|---|---|---|
| Non-DC Xylitol | 50 | — | — | — | — | — |
| Non-DC Lactitol | — | 50 | — | — | — | — |
| Non-DC Isomalt | — | — | 50 | — | — | — |
| Non-DC Erythritol | — | — | — | 50 | — | — |
| Non-DC Mannitol | — | — | — | — | 50 | — |
| Non-DC Maltitol | — | — | — | — | — | 50 |
| Acetaminophen** | 46 | 46 | 46 | 46 | 46 | 46 |
| Flavor* | 4 | 4 | 4 | 4 | 4 | 4 |

*powder flavor.
**Including a granulation of 50% acetaminophen, 23% microcrystalline cellulose (MCC), 22% mannitol and 5% starch (by weight).

TABLE 5

| Raw material (wt %) | Ex7A | Ex7B | Ex7C | Ex7D | Ex7E | Ex7F |
|---|---|---|---|---|---|---|
| Non-DC Xylitol | 81 | 86 | 76 | 66 | 56 | 36 |
| Acetaminophen** | 5 | 10 | 20 | 30 | 40 | 60 |
| Flavor* | 4 | 4 | 4 | 4 | 4 | 4 |

*powder flavor.
**Including a granulation of 50% acetaminophen, 23% microcrystalline cellulose (MCC), 22% mannitol and 5% starch (by weight).

TABLE 6

| Raw material (wt %) | Ex7G | Ex7H | Ex1I | Ex7J | Ex7K | Ex7L |
|---|---|---|---|---|---|---|
| Non-DC Xylitol | 39 | 38 | 36 | 35 | 32 | 30 |
| Acetaminophen** | 60 | 60 | 60 | 60 | 60 | 60 |
| Flavor* | 1 | 2 | 4 | 5 | 8 | 10 |

*powder flavor.
**Including a granulation of 50% acetaminophen, 23% microcrystalline cellulose (MCC), 22% mannitol and 5% starch (by weight).

TABLE 7

| Raw material (wt %) | Ex13 | Ex14 | Ex15 | Ex16 | Ex17 |
|---|---|---|---|---|---|
| Dextrose | 50 | — | — | — | — |
| Saccharose | — | 50 | — | — | — |
| Dextrin | — | — | 50 | — | — |
| Trehalose | — | — | — | 50 | — |
| Fructose | — | — | — | — | 50 |
| Acetaminophen** | 46 | 46 | 46 | 46 | 46 |
| Flavor* | 4 | 4 | 4 | 4 | 4 |

*powder flavor.
**Including a granulation of 50% acetaminophen, 23% microcrystalline cellulose (MCC), 22% dextrose and 5% starch (by weight).

TABLE 8

| Raw material | | | | | | |
|---|---|---|---|---|---|---|
| (wt %) | Ex13A | Ex13B | Ex13C | Ex13D | Ex13E | Ex13F |
| Dextrose | 81 | 86 | 76 | 66 | 56 | 36 |
| Acetamin- | 5 | 10 | 20 | 30 | 40 | 60 |
| ophen** | | | | | | |
| Flavor* | 4 | 4 | 4 | 4 | 4 | 4 |

*powder flavor.
**Including a granulation of 50% acetaminophen, 23% microcrystalline cellulose (MCC), 22% dextrose and 5% starch (by weight).

TABLE 9

| Raw material | | | | | | |
|---|---|---|---|---|---|---|
| (wt %) | Ex13G | Ex13H | Ex13I | Ex13J | Ex13K | Ex13L |
| Dextrose | 39 | 38 | 36 | 35 | 32 | 30 |
| Acetamin- | 60 | 60 | 60 | 60 | 60 | 60 |
| ophen** | | | | | | |
| Flavor* | 1 | 2 | 4 | 5 | 8 | 10 |

*powder flavor.
**Including a granulation of 50% acetaminophen, 23% microcrystalline cellulose (MCC), 22% dextrose and 5% starch (by weight).

TABLE 10

| Raw material (wt %) | Ex18 | Ex19 | Ex20 | Ex21 | Ex22 | Ex23 |
|---|---|---|---|---|---|---|
| DC Xylitol | 89 | — | — | — | — | — |
| Sorbitol | — | 89 | — | — | — | — |
| DC Isomalt | — | — | 89 | — | — | — |
| DC Erythritol | — | — | — | 89 | — | — |
| DC Mannitol | — | — | — | — | 89 | — |
| DC Maltitol | — | — | — | — | — | 89 |
| Phenylephrine** | 5 | 5 | 5 | 5 | 5 | 5 |
| Flavor* | 6 | 6 | 6 | 6 | 6 | 6 |

*powder flavor.
**Including a granulation of 10% phenylephrine HCl, 44% microcrystalline cellulose (MCC), 41% sorbitol and 5% starch (by weight).

TABLE 11

| Raw material | | | | | | |
|---|---|---|---|---|---|---|
| (wt %) | Ex18A | Ex19A | Ex20A | Ex21A | Ex22A | Ex23A |
| DC Xylitol | 50 | — | — | — | — | — |
| Sorbitol | — | 50 | — | — | — | — |
| DC Isomalt | — | — | 50 | — | — | — |
| DC Erythritol | — | — | — | 50 | — | — |
| DC Mannitol | — | — | — | — | 50 | — |
| DC Maltitol | — | — | — | — | — | 50 |
| Phenyl- | 46 | 46 | 46 | 46 | 46 | 46 |
| ephrine** | | | | | | |
| Flavor* | 4 | 4 | 4 | 4 | 4 | 4 |

*powder flavor.
**Including a granulation of 1% phenylephrine HCl, 47% microcrystalline cellulose (MCC), 45% mannitol and 7% starch (by weight).

TABLE 12

| Raw material (wt %) | Ex24 | Ex25 | Ex26 | Ex27 | Ex28 | Ex29 |
|---|---|---|---|---|---|---|
| Non-DC Xylitol | 89 | — | — | — | — | — |
| Non-DC Lactitol | — | 89 | — | — | — | — |
| Non-DC Isomalt | — | — | 89 | — | — | — |
| Non-DC Erythritol | — | — | — | 89 | — | — |
| Non-DC Mannitol | — | — | — | — | 89 | — |
| Non-DC Maltitol | — | — | — | — | — | 89 |
| Phenylephrine** | 5 | 5 | 5 | 5 | 5 | 5 |
| Flavor* | 6 | 6 | 6 | 6 | 6 | 6 |

*powder flavor.
**Including a granulation of 10% phenylephrine HCl, 44% microcrystalline cellulose (MCC), 41% mannitol and 5% starch (by weight).

TABLE 13

| Raw material (wt %) | Ex24A | Ex25A | Ex26A | Ex27A | Ex28A | Ex29A |
|---|---|---|---|---|---|---|
| Non-DC Xylitol | 50 | — | — | — | — | — |
| Non-DC Lactitol | — | 50 | — | — | — | — |
| Non-DC Isomalt | — | — | 50 | — | — | — |
| Non-DC Erythritol | — | — | — | 50 | — | — |
| Non-DC Mannitol | — | — | — | — | 50 | — |
| Non-DC Maltitol | — | — | — | — | — | 50 |
| Phenylephrine** | 46 | 46 | 46 | 46 | 46 | 46 |
| Flavor* | 4 | 4 | 4 | 4 | 4 | 4 |

*powder flavor.
**Including a granulation of 1% phenylephrine HCl, 47% microcrystalline cellulose (MCC), 45% sorbitol and 7% starch (by weight).

TABLE 14

| Raw material (wt %) | Ex30 | Ex31 | Ex32 | Ex33 | Ex34 |
|---|---|---|---|---|---|
| Dextrose | 89 | — | — | — | — |
| Saccharose | — | 89 | — | — | — |
| Dextrin | — | — | 89 | — | — |
| Trehalose | — | — | — | 89 | — |
| Fructose | — | — | — | — | 89 |
| Phenylephrine** | 5 | 5 | 5 | 5 | 5 |
| Flavor* | 6 | 6 | 6 | 6 | 6 |

*powder flavor.
**Including a granulation of 10% phenylephrine HCl, 44% microcrystalline cellulose (MCC), 41% dextrose and 5% starch (by weight).

TABLE 15

| Raw material (wt %) | Ex30A | Ex31A | Ex32A | Ex33A | Ex34A |
|---|---|---|---|---|---|
| Dextrose | 50 | — | — | — | — |
| Saccharose | — | 50 | — | — | — |
| Dextrin | — | — | 50 | — | — |
| Trehalose | — | — | — | 50 | — |
| Fructose | — | — | — | — | 50 |
| Phenylephrine** | 46 | 46 | 46 | 46 | 46 |
| Flavor* | 4 | 4 | 4 | 4 | 4 |

*powder flavor.
**Including a granulation of 1% phenylephrine HCl, 47% microcrystalline cellulose (MCC), 45% dextrose and 7% starch (by weight).

TABLE 16

| Raw material (wt %) | Ex35 | Ex36 | Ex37 | Ex38 | Ex39 | Ex40 |
|---|---|---|---|---|---|---|
| DC Xylitol | 82 | — | — | — | — | — |
| Sorbitol | — | 82 | — | — | — | — |
| DC Isomalt | — | — | 82 | — | — | — |
| DC Erythritol | — | — | — | 82 | — | — |
| DC Mannitol | — | — | — | — | 82 | — |
| DC Maltitol | — | — | — | — | — | 82 |
| Dextromethorphan** | 10 | 10 | 10 | 10 | 10 | 10 |
| Flavor* | 8 | 8 | 8 | 8 | 8 | 8 |

*powder flavor.
**Including a granulation of 10% dextromethorphan, 45% microcrystalline cellulose (MCC), 40% sorbitol and 5% starch (by weight).

TABLE 17

| Raw material (wt %) | Ex35A | Ex36A | Ex37A | Ex38A | Ex39A | Ex40A |
|---|---|---|---|---|---|---|
| DC Xylitol | 50 | — | — | — | — | — |
| Sorbitol | — | 50 | — | — | — | — |
| DC Isomalt | — | — | 50 | — | — | — |
| DC Erythritol | — | — | — | 50 | — | — |
| DC Mannitol | — | — | — | — | 50 | — |
| DC Maltitol | — | — | — | — | — | 50 |
| Dextromethorphan** | 46 | 46 | 46 | 46 | 46 | 46 |
| Flavor* | 4 | 4 | 4 | 4 | 4 | 4 |

*powder flavor.
**Including a granulation of 1% dextromethorphan, 47% microcrystalline cellulose (MCC), 45% sorbitol and 7% starch (by weight).

TABLE 18

| Raw material (wt %) | Ex41 | Ex42 | Ex43 | Ex44 | Ex45 | Ex46 |
|---|---|---|---|---|---|---|
| Non-DC Xylitol | 82 | — | — | — | — | — |
| Non-DC Lactitol | — | 82 | — | — | — | — |
| Non-DC Isomalt | — | — | 82 | — | — | — |
| Non-DC Erythritol | — | — | — | 82 | — | — |
| Non-DC Mannitol | — | — | — | — | 82 | — |
| Non-DC Maltitol | — | — | — | — | — | 82 |
| Dextromethorphan** | 10 | 10 | 10 | 10 | 10 | 10 |
| Flavor* | 8 | 8 | 8 | 8 | 8 | 8 |

*powder flavor.
**Including a granulation of 10% dextromethorphan, 45% microcrystalline cellulose (MCC), 40% mannitol and 5% starch (by weight).

TABLE 19

| Raw material (wt %) | Ex41A | Ex42A | Ex43A | Ex44A | Ex45A | Ex46A |
|---|---|---|---|---|---|---|
| Non-DC Xylitol | 50 | — | — | — | — | — |
| Non-DC Lactitol | — | 50 | — | — | — | — |
| Non-DC Isomalt | — | — | 50 | — | — | — |
| Non-DC Erythritol | — | — | — | 50 | — | — |
| Non-DC Mannitol | — | — | — | — | 50 | — |
| Non-DC Maltitol | — | — | — | — | — | 50 |
| Dextromethorphan** | 46 | 46 | 46 | 46 | 46 | 46 |
| Flavor* | 4 | 4 | 4 | 4 | 4 | 4 |

*powder flavor.
**Including a granulation of 1% dextromethorphan, 47% microcrystalline cellulose (MCC), 45% mannitol and 7% starch (by weight).

TABLE 20

| Raw material (wt %) | Ex47 | Ex48 | Ex49 | Ex50 | Ex51 |
|---|---|---|---|---|---|
| Dextrose | 82 | — | — | — | — |
| Saccharose | — | 82 | — | — | — |
| Dextrin | — | — | 82 | — | — |
| Trehalose | — | — | — | 82 | — |
| Fructose | — | — | — | — | 82 |
| Dextromethorphan ** | 10 | 10 | 10 | 10 | 10 |
| Flavor* | 8 | 8 | 8 | 8 | 8 |

*powder flavor.
** Including a granulation of 10% dextromethorphan, 45% microcrystalline cellulose (MCC), 40% dextrose and 5% starch (by weight).

TABLE 21

| Raw material (wt %) | Ex47A | Ex48A | Ex49A | Ex50A | Ex51A |
|---|---|---|---|---|---|
| Dextrose | 50 | — | — | — | — |
| Saccharose | — | 50 | — | — | — |
| Dextrin | — | — | 50 | — | — |
| Trehalose | — | — | — | 50 | — |
| Fructose | — | — | — | — | 50 |
| Dextromethorphan ** | 46 | 46 | 46 | 46 | 46 |
| Flavor* | 4 | 4 | 4 | 4 | 4 |

*powder flavor.
** Including a granulation of 1% dextromethorphan, 47% microcrystalline cellulose (MCC), 45% dextrose and 7% starch (by weight).

TABLE 22

| Raw material (wt %) | Ex52 | Ex53 | Ex54 | Ex55 | Ex56 | Ex57 |
|---|---|---|---|---|---|---|
| Non-DC Xylitol | 50 | — | — | — | — | — |
| Non-DC Lactitol | — | 50 | — | — | — | — |
| Non-DC Isomalt | — | — | 50 | — | — | — |
| Non-DC Erythritol | — | — | — | 50 | — | — |
| Non-DC Mannitol | — | — | — | — | 50 | — |
| Non-DC Maltitol | — | — | — | — | — | 50 |
| First type of particles** | 46 | 46 | 46 | 46 | 46 | 46 |
| Flavor* | 4 | 4 | 4 | 4 | 4 | 4 |

*powder flavor.
**Including distinct portions of granules where one distinct portion of granules contains 1% phenylephrine HCl, another portion 1% dextromethorphan, and yet another portion 48% acetaminophen, all portions including microcrystalline cellulose (MCC), sorbitol and starch in the same amounts as for the distinct granules as outlined in the previous examples with the same amounts of API (by weight).

TABLE 23

| Raw material (wt %) | Ex58 | Ex59 | Ex60 | Ex61 | Ex62 |
|---|---|---|---|---|---|
| Dextrose | 50 | — | — | — | — |
| Saccharose | — | 50 | — | — | — |
| Dextrin | — | — | 50 | — | — |
| Trehalose | — | — | — | 50 | — |
| Fructose | — | — | — | — | 50 |
| First type of | 46 | 46 | 46 | 46 | 46 |
| particles** | | | | | |
| Flavor* | 4 | 4 | 4 | 4 | 4 |

*powder flavor.
**Including distinct portions of granules where one distinct portion of granules contains 1% phenylephrine HCl, another portion 1% dextromethorphan, and yet another portion 48% acetaminophen, all portions including microcrystalline cellulose (MCC), dextrose and starch in the same amounts as for the distinct granules as outlined in the previous examples with the same amounts of API (by weight).

TABLE 24

| Raw material (wt %) | Ex63 | Ex64 | Ex65 | Ex66 | Ex67 | Ex68 |
|---|---|---|---|---|---|---|
| Non-DC Xylitol | 25 | — | 25 | — | — | — |
| Non-DC Mannitol | — | 25 | — | 25 | — | — |
| DC Xylitol | 25 | — | — | — | 25 | — |
| Sorbitol | — | 25 | — | — | — | 25 |
| Dextrose | — | — | 25 | — | 25 | — |
| Fructose | — | — | — | 25 | — | 25 |
| First type of particles** | 46 | 46 | 46 | 46 | 46 | 46 |
| Flavor* | 4 | 4 | 4 | 4 | 4 | 4 |

*powder flavor.
**Including distinct portions of granules where one distinct portion of granules contains 1% phenylephrine HCl, another portion 1% dextromethorphan, and yet another portion 48% acetaminophen, all portions including microcrystalline cellulose (MCC), mannitol and starch in the same amounts as for the distinct granules as outlined in the previous examples with the same amounts of API (by weight).

### Process Flow

For each example the raw materials are sieved with a 1600 micron sieve and then weighed into the proper amount according to the above-outlined compositions.

The weighed amounts are then added to a Turbula mixer in a stainless-steel container and blended at 50 rpm for 5 minutes. MgSt was optionally added after 4 minutes of blending. A glidant such as silicon dioxide may optionally be added at this step too.

An amount of 1200 mg powder was tested with respect to various sensorial parameters, including taste masking, mouthfeel and overall liking.

These powder samples were each compared with corresponding powders made into compressed tablets. The comparative tablets were tableted by means of a Piccola RIVA DC-SC-041-2 or a Fette 3090i. The applied molds have circular cross sections with diameters of 16 mm and are hollowed to produce tablets, which are concave and/or curved. Evidently, other mold size and shapes may be applied within the scope of the invention. The resulting comparative tablets were then obtained by tableting with a suitable pressure force (approx. 20 kN).

A specification of relevant compounds applied in the examples explained above are listed below.
HPC: Hydroxy propyl cellulose. Klucel Nutra D from Ashland
Non-DC Xylitol: Xivia C from Dupont
Non-DC Isomalt: Isomalt GS from Beneo Paltinit
Non-DC Mannitol: C*PharmMannidex 16700 from Cargill
Non-DC Maltitol: Maltisorb. P200 from Roquette
Non-DC Erythritol: Zerose 16952 from Cargill
Non-DC Lactitol: from Dupont
DC Erythritol—Zerose 16966 from Cargill
Sorbitol: Neosorb P 300 C from Roquette
DC Xylitol—Xylitab 200 from Dupont
DC Isomalt—Isomalt DC 101 from Beneo Paltinit
DC Mannitol—Pearlitol 200 SD from Roquette
DC Maltitol—Sweetpearl 300 DC from Roquette
Fructose: Fructose M from ADM
Dextrose: C*PharmDex02011 from Cargill
Saccharose: Saccharose from Nordzucker
Dextrin: Tackidex (Potato Dextrin) from Roquette
Thehalose: Biohale (Trehalose Dihydrate) from DFE Pharma
Microcrystalline cellulose (MCC): Avicel PH101 from FMC
Lactose: Pharmatose 200M from DFE Pharma
Starch: Starch 1500® from Colorcon
Magnesium stearate: MF-2-V from Peter Greven
Silicon dioxide: Aerosil 200 from Evonik
Phenylephrine HCl: Batch no. P70-19001 from Syn-Tech
Dextromethorphan: Batch no. EVS11884 from Wockhardt
Acetaminophen: Batch no. 180830C1 from Novacyl

The procedure of granulation of the active pharmaceutical ingredients was as follows:

The various ingredients for use in the granules were weighted in specified amounts and sieved through a 1 mm sieve before being added to a high shear mixer available from Diosna. This mixture was mixed at a speed of about 250 rpm (impeller speed) and about 300 rpm (chopper speed) for about 3 minutes. The loss on drying (LOD) of the blend was about 3% after this step.

Thereafter, the mixture was wetted and granulated with purified water being slowly added to give a wetting percentage of about 20-30%. Here, the blend was processed with a speed of about 300 rpm (impeller speed) and about 500 rpm (chopper speed) for about 15 minutes. The wet granules were screened through a 2 mm screen using an oscillating calibrator available from ERWEKA.

Then the screened wet granules were dried using a fluidized bed device available from Glatt. The inlet temperature was about 50 Degree Celsius with an air flow rate of above 40 m3/h, a product temperature of about 28 Degree Celsius and an outlet temperature of about 28 Degree Celsius. The dried granular particles had a loss on drying (LOD) of approx. 3%. Subsequently, the dried granules were sieved through a 1 mm sieve and stored.

### Evaluation

### Sensoric Evaluation Test Set-Up

The test set-up was composed of 8 test persons in a test panel. Each of the test persons were healthy individuals appointed on an objective basis according to specified requirements. The sensory analysis was performed according to ISO 4121-2003 in testing conditions following ISO 8589. The result is an average of the results of the 8 individuals.

The test persons gave a rating from “1” to “5”, where “1” is poor and “5” is excellent.

When referring to “initial watering effect” (increased saliva generation), “mouthfeel”, “taste masking”, and “liking score”, the tests were made using the following method, unless stated otherwise.

Test subjects abstain from eating and drinking at least 30 minutes before initiation of any test. Immediately before introducing of the powder sample or tablet into the oral cavity, the test subject swallows. The test subject refrains from swallowing during the test. Immediately after introducing of the tablet into the oral cavity, the test subject starts masticating the tablet at a frequency of 1 chew per second for 20 seconds. In case of powder samples, the test subject moves the sample around using the tongue at a frequency of 1 back and forth per second for 20 seconds. Then, saliva and any remains of the powder sample or tablet is kept in the mouth for 10 seconds. During this period, the test subject evaluates the sensorial parameters “mouth feel”, “taste masking” and “liking”. 30 seconds after starting the test, the test subject discards saliva including any powder or tablet fragments into a plastic cup, which is weighted. Saliva discarded also at 90 seconds after onset of mastication, at 180 seconds after onset of mastication, at 300 seconds after onset of mastication, at 420 seconds after onset of mastication, and at 600 seconds after onset of mastication. At all times, the test subject makes as little movement as possible, and refrains from swallowing.

### Evaluation

### Results

All samples were evaluated and found to be similar to the exemplary evaluations set forth in the table below:

TABLE 25

| | Overall | | | | | |
|---|---|---|---|---|---|---|
| | sensory | | Initial | | | |
| | experience | | Watering | | Taste | Liking |
| | Very | | effect | Mouthfeel | masking | score |
| | good/Good/ | | 1-5 | 1-5 | 1-5 | 1-5 |
| | Acceptable | Dosage | 1 low | 1 low | 1 low | 1 low |
| Ex | (Acc)/Poor | form | 5 high | 5 high | 5 high | 5 high |
| 2 | Very good | Powder | 5 | 5 | 5 | 5 |
| 2* | Poor | Tablet | 2 | 1 | 1 | 2 |
| 7 | Very good | Powder | 5 | 4 | 4 | 5 |
| 7* | Good | Tablet | 4 | 4 | 3 | 4 |
| 11 | Very good | Powder | 5 | 5 | 5 | 5 |
| 11* | Good | Tablet | 4 | 4 | 3 | 3 |
| 13 | Very good | Powder | 5 | 5 | 5 | 5 |
| 13* | Good | Tablet | 3 | 4 | 4 | 4 |

Sensory evaluation of selected examples. Scores shown are averages of scores from the individual panelists *examples 2, 7, 11 and 13 were compared to the same powders made into compressed tablets.

The above powders and tablets were evaluated according to four parameters by a test panel.

One of the parameters was the overall sensory experience as a powder or tablet and one of the parameters was the perceived watering effect.

It was first of all noted that the watering effect was considered very high for the inventive examples. The watering effect is considered to be representative or equal to the elsewhere described salivation effect (or generation of saliva). This was highly surprising results.

The test panel clearly indicated that the overall mouthfeel was no less than impressive in relation to various of the inventive examples. It was also noted that the test panel identified inventive examples an impressive watering effect when compared to e.g. the tablet-based example.

With respect to taste-making, impressive results were seen compared to tablets which was contrary to every expectation of the inventors. Generally, it was expected that the powders would incur much more pronounced taste issues but the opposite occurred. The powders showed an increased taste masking effect compared to the tablets. This was highly surprising. Equally surprising was that the “liking” score showed even more pronounced results.

1. A flowpack for oral delivery of active pharmaceutical ingredients, the flowpack comprising a flowable population of particles comprising:
a first type of particles comprising one or more distinct portions of particles with a content of one or more active pharmaceutical ingredients and a carrier for the one or more active pharmaceutical ingredients;
a second type of particles in an amount of 30-95% by weight of the population of particles separate from the first type of particles, the second type of particles comprising at least 95% by weight of one or more sweeteners; and
a third type of particles in an amount of 1-10% by weight of the population of particles separate from the first and second type of particles, the third type of particles comprising flavor.

2. The flowpack according to claim 1, wherein the flowable population of particles is a free-flowing population of particles.

3. The flowpack according any one of claim 1 or 2, wherein the flowable population of particles constitutes more than 70% by weight of the flowpack.

4. The flowpack according any one of the preceding claims, wherein the flowable population of particles constitutes more than 90% by weight of the flowpack.

5. The flowpack according any one of the preceding claims, wherein the flowable population of particles constitutes more than 95% by weight of the flowpack.

6. The flowpack according any one of the preceding claims, wherein the first, second and third type of particles constitutes more than 70% by weight of the flowable population of particles.

7. The flowpack according any one of the preceding claims, wherein the first, second and third type of particles constitutes more than 90% by weight of the flowable population of particles.

8. The flowpack according any one of the preceding claims, wherein the first, second and third type of particles constitutes more than 99% by weight of the flowable population of particles.

9. The flowpack according to any one of the preceding claims, wherein the second type of particles is present in an amount of 35-95% by weight of the population of particles.

10. The flowpack according to any one of the preceding claims, wherein the second type of particles is present in an amount of 35-80% by weight of the population of particles.

11. The flowpack according to any one of the preceding claims, wherein the second type of particles is present in an amount of 40-60% by weight of the population of particles.

12. The flowpack according to any one of the preceding claims, wherein the second type of particles is present in an amount of 20-40% by weight of the population of particles.

13. The flowpack according to any one of the preceding claims, wherein an average particle size of the second type of particles is between 50 and 500 microns.

14. The flowpack according to any one of the preceding claims, wherein an average particle size of the second type of particles is between 100 and 400 microns.

15. The flowpack according to any one of the preceding claims, wherein a series of at least 5 samples of the second type of particles each having the same fixed weight in the range of 0.5-2 g varying with a relative standard deviation (RSD) below 10%.

16. The flowpack according to any one of the preceding claims, wherein a series of at least 5 samples of the second type of particles each having the same fixed weight in the range of 0.5-2 g varying with a relative standard deviation (RSD) below 5%.

17. The flowpack according to any one of the preceding claims, wherein the second type of particles comprises at least 98% by weight of one or more sweeteners.

18. The flowpack according to any one of the preceding claims, wherein the one or more sweeteners of the second type of particles comprises directly compressible (DC) sweetener particles.

19. The flowpack according to any one of the preceding claims, wherein the one or more sweeteners of the second type of particles comprises non-directly compressible (non-DC) sweetener particles.

20. The flowpack according to any one of the preceding claims, wherein the one or more sweeteners of the second type of particles comprises non-directly compressible (non-DC) sweetener particles and directly compressible (DC) sweetener particles in a ratio of 0.2 and 1.2.

21. The flowpack according to any one of the preceding claims, wherein the one or more sweeteners of the second type of particles comprises non-directly compressible (non-DC) sweetener particles and directly compressible (DC) sweetener particles in a ratio of 0.3 and 0.7.

22. The flowpack according to any one of the preceding claims, wherein the one or more sweeteners of the second type of particles comprises one or more sugar alcohols.

23. The flowpack according to any one of the preceding claims, wherein the one or more sweeteners of the second type of particles comprises one or more sugar alcohols selected from the group consisting of erythritol, maltitol, xylitol, isomalt, lactitol, mannitol, sorbitol, and combinations thereof.

24. The flowpack according to any one of the preceding claims, wherein the one or more sweeteners of the second type of particles comprises mannitol.

25. The flowpack according to any one of the preceding claims, wherein the one or more sweeteners of the second type of particles comprises one or more saccharides.

26. The flowpack according to any one of the preceding claims, wherein the one or more sweeteners of the second type of particles comprises one or more saccharides selected from the group consisting of dextrose, saccharose, dextrin, trehalose, fructose, and combinations thereof.

27. The flowpack according to any one of the preceding claims, wherein the one or more sweeteners of the second type of particles comprises dextrose.

28. The flowpack according to any one of the preceding claims, wherein the third type of particles is present in an amount of 1-8% by weight of the population of particles.

29. The flowpack according to any one of the preceding claims, wherein the third type of particles is present in an amount of 1-5% by weight of the population of particles.

30. The flowpack according to any one of the preceding claims, wherein the third type of particles is present in an amount of 2-5% by weight of the population of particles.

31. The flowpack according to any one of the preceding claims, wherein an average particle size of the third type of particles is between 50 and 500 microns.

32. The flowpack according to any one of the preceding claims, wherein an average particle size of the third type of particles is between 100 and 400 microns.

33. The flowpack according to any one of the preceding claims, wherein a series of at least 5 samples of the third type of particles each having the same fixed weight in the range of 0.5-2 g varying with a relative standard deviation (RSD) below 10%.

34. The flowpack according to any one of the preceding claims, wherein a series of at least 5 samples of the third type of particles each having the same fixed weight in the range of 0.5-2 g varying with a relative standard deviation (RSD) below 5%.

35. The flowpack according to any one of the preceding claims, wherein the third type of particles comprises a powder flavor.

36. The flowpack according to any one of the preceding claims, wherein the third type of particles is a powder flavor.

37. The flowpack according to any one of the preceding claims, wherein the third type of particles has a load of flavor in a range of 5-25%.

38. The flowpack according to any one of the preceding claims, wherein the flavor of the third type of particles is selected from the group consisting of coconut, coffee, vanilla, blackcurrant, lemon, grapefruit, orange, lime, menthol, liquorice, caramel aroma, honey aroma, pineapple, strawberry, raspberry, tropical fruits, cherries, cinnamon, peppermint, wintergreen, spearmint, eucalyptus, mint, fruit essence, creme essence, and combinations thereof.

39. The flowpack according to any one of the preceding claims, wherein one or more distinct portions of particles of the first type of particles is present in an amount of 30-60% by weight of the population of particles.

40. The flowpack according to any one of the preceding claims, wherein one or more distinct portions of particles of the first type of particles is present in an amount of 40-60% by weight of the population of particles.

41. The flowpack according to any one of the preceding claims, wherein one or more distinct portions of particles of the first type of particles is present in an amount of 10-40% by weight of the population of particles.

42. The flowpack according to any one of the preceding claims, wherein one or more distinct portions of particles of the first type of particles is present in an amount of 20-40% by weight of the population of particles.

43. The flowpack according to any one of the preceding claims, wherein one or more distinct portions of particles of the first type of particles is present in an amount of 2-20% by weight of the population of particles.

44. The flowpack according to any one of the preceding claims, wherein one or more distinct portions of particles of the first type of particles is present in an amount of 2-10% by weight of the population of particles.

45. The flowpack according to any one of the preceding claims, wherein an average particle size of the first type of particles is between 50 and 500 microns.

46. The flowpack according to any one of the preceding claims, wherein an average particle size of the first type of particles is between 100 and 400 microns.

47. The flowpack according to any one of the preceding claims, wherein an average particle size of the first type of particles is between 200 and 400 microns.

48. The flowpack according to any one of the preceding claims, wherein an average particle size of the first type of particles is between 400 and 600 microns.

49. The flowpack according to any one of the preceding claims, wherein a series of at least 5 samples of the first type of particles each having the same fixed weight in the range of 0.5-2 g varying with a relative standard deviation (RSD) below 10%.

50. The flowpack according to any one of the preceding claims, wherein a series of at least 5 samples of the first type of particles each having the same fixed weight in the range of 0.5-2 g varying with a relative standard deviation (RSD) below 5%.

51. The flowpack according to any one of the preceding claims, wherein the one or more distinct portions of particles of the first type of particles comprises one or more low dose active pharmaceutical ingredients.

52. The flowpack according to any one of the preceding claims, wherein the one or more distinct portions of particles of the first type of particles comprises one or more low dose active pharmaceutical ingredients in an amount of less than 4% by weight of the population of particles.

53. The flowpack according to any one of the preceding claims, wherein the one or more distinct portions of particles of the first type of particles comprises one or more low dose active pharmaceutical ingredients in an amount of less than 2% by weight of the population of particles.

54. The flowpack according to any one of the preceding claims, wherein the one or more distinct portions of particles of the first type of particles comprises one or more low dose active pharmaceutical ingredients selected from the group consisting of diphenhydramine, cetirizine, loratadine, chlorpheniramine maleate, levocetirizine, meclizine, dextromethorphan, phenylephrine, famotidine, omeprazole, doxylamine succinate, melatonin, and any combination and mixture thereof.

55. The flowpack according to any one of the preceding claims, wherein the one or more distinct portions of particles of the first type of particles comprises one or more low dose antihistamine ingredients selected from the group consisting of diphenhydramine, cetirizine, loratadine, chlorpheniramine maleate, levocetirizine, and any combination and mixture thereof.

56. The flowpack according to any one of the preceding claims, wherein the one or more distinct portions of particles of the first type of particles comprises one or more low dose cough suppressant ingredients comprising dextromethorphan.

57. The flowpack according to any one of the preceding claims, wherein the one or more distinct portions of particles of the first type of particles comprises one or more low dose motion sickness ingredients comprising meclizine.

58. The flowpack according to any one of the preceding claims, wherein the one or more distinct portions of particles of the first type of particles comprises one or more low dose decongestant ingredients comprising phenylephrine.

59. The flowpack according to any one of the preceding claims, wherein the one or more distinct portions of particles of the first type of particles comprises one or more low dose heartburn ingredients selected from the group consisting of famotidine, omeprazole, and any combination and mixture thereof.

60. The flowpack according to any one of the preceding claims, wherein the one or more distinct portions of particles of the first type of particles comprises one or more low dose insomnia ingredients selected from the group consisting of doxylamine succinate, melatonin, and any combination and mixture thereof.

61. The flowpack according to any one of the preceding claims, wherein the one or more distinct portions of particles of the first type of particles comprises one or more low dose active pharmaceutical ingredients present in an amount of 2-30 mg in the flow pack.

62. The flowpack according to any one of the preceding claims, wherein the one or more distinct portions of particles of the first type of particles comprises one or more low dose active pharmaceutical ingredients present in an amount of 2-10 mg in the flow pack.

63. The flowpack according to any one of the preceding claims, wherein the one or more distinct portions of particles of the first type of particles comprising the one or more low dose active pharmaceutical ingredients is present in an amount of 2-20% by weight of the population of particles.

64. The flowpack according to any one of the preceding claims, wherein the one or more distinct portions of particles of the first type of particles comprising the one or more low dose active pharmaceutical ingredients is present in an amount of 2-10% by weight of the population of particles.

65. The flowpack according to any one of the preceding claims, wherein the one or more distinct portions of particles of the first type of particles comprises one or more medium dose active pharmaceutical ingredients.

66. The flowpack according to any one of the preceding claims, wherein the one or more distinct portions of particles of the first type of particles comprises one or more medium dose active pharmaceutical ingredients in an amount of 4 to 30% by weight of the population of particles.

67. The flowpack according to any one of the preceding claims, wherein the one or more distinct portions of particles of the first type of particles comprises one or more medium dose active pharmaceutical ingredients in an amount of 4 to 20% by weight of the population of particles.

68. The flowpack according to any one of the preceding claims, wherein the one or more distinct portions of particles of the first type of particles comprises one or more medium dose active pharmaceutical ingredients selected from the group consisting of diphenhydramine, fexofenadine, dimenhydrinate, meclizine, pseudoephedrine, aspirin, caffeine, theanine, and any combination and mixture thereof.

69. The flowpack according to any one of the preceding claims, wherein the one or more distinct portions of particles of the first type of particles comprises one or more medium dose antihistamine ingredients selected from the group consisting of diphenhydramine, fexofenadine, and any combination and mixture thereof.

70. The flowpack according to any one of the preceding claims, wherein the one or more distinct portions of particles of the first type of particles comprises one or more medium dose motion sickness ingredients selected from the group consisting of dimenhydrinate, meclizine, and any combination and mixture thereof.

71. The flowpack according to any one of the preceding claims, wherein the one or more distinct portions of particles of the first type of particles comprises one or more medium dose decongestant ingredients comprising pseudoephedrine.

72. The flowpack according to any one of the preceding claims, wherein the one or more distinct portions of particles of the first type of particles comprises one or more medium dose analgesics comprising aspirin.

73. The flowpack according to any one of the preceding claims, wherein the one or more distinct portions of particles of the first type of particles comprises one or more medium dose alertness ingredients selected from the group consisting of caffeine, theanine, and any combination and mixture thereof.

74. The flowpack according to any one of the preceding claims, wherein the one or more distinct portions of particles of the first type of particles comprises one or more medium dose active pharmaceutical ingredients in an amount of 20-150 mg in the flow pack.

75. The flowpack according to any one of the preceding claims, wherein the one or more distinct portions of particles of the first type of particles comprises one or more medium dose active pharmaceutical ingredients in an amount of 30-100 mg in the flow pack.

76. The flowpack according to any one of the preceding claims, wherein the one or more distinct portions of particles of the first type of particles comprising the one or more medium dose active pharmaceutical ingredients is present in an amount of 10-40% by weight of the population of particles.

77. The flowpack according to any one of the preceding claims, wherein the one or more distinct portions of particles of the first type of particles comprising the one or more medium dose active pharmaceutical ingredients is present in an amount of 20-40% by weight of the population of particles.

78. The flowpack according to any one of the preceding claims, wherein the one or more distinct portions of particles of the first type of particles comprises one or more high dose active pharmaceutical ingredients.

79. The flowpack according to any one of the preceding claims, wherein the one or more distinct portions of particles of the first type of particles comprises one or more high dose active pharmaceutical ingredients in an amount of 20 to 70% by weight of the population of particles.

80. The flowpack according to any one of the preceding claims, wherein the one or more distinct portions of particles of the first type of particles comprises one or more high dose active pharmaceutical ingredients in an amount of 20 to 50% by weight of the population of particles.

81. The flowpack according to any one of the preceding claims, wherein the one or more distinct portions of particles of the first type of particles comprises one or more high dose active pharmaceutical ingredients selected from the group consisting of fexofenadine, guaifenesin, calcium carbonate, magnesium hydroxide, acetaminophen, aspirin, ibuprofen, naproxen sodium, caffeine, and any combination and mixture thereof.

82. The flowpack according to any one of the preceding claims, wherein the one or more distinct portions of particles of the first type of particles comprises one or more high dose antihistamine ingredients comprising fexofenadine.

83. The flowpack according to any one of the preceding claims, wherein the one or more distinct portions of particles of the first type of particles comprises one or more high dose expectorant ingredients comprising guaifenesin.

84. The flowpack according to any one of the preceding claims, wherein the one or more distinct portions of particles of the first type of particles comprises one or more high dose antacid ingredients selected from the group consisting of calcium carbonate, magnesium hydroxide, and any combination and mixture thereof.

85. The flowpack according to any one of the preceding claims, wherein the one or more distinct portions of particles of the first type of particles comprises one or more high dose analgesics ingredients selected from the group consisting of acetaminophen, aspirin, ibuprofen, naproxen sodium, and any combination and mixture thereof.

86. The flowpack according to any one of the preceding claims, wherein the one or more distinct portions of particles of the first type of particles comprises one or more high dose alertness ingredients comprising caffeine.

87. The flowpack according to any one of the preceding claims, wherein the one or more distinct portions of particles of the first type of particles comprises one or more high dose active pharmaceutical ingredients in an amount of 150-1000 mg in the flow pack.

88. The flowpack according to any one of the preceding claims, wherein the one or more distinct portions of particles of the first type of particles comprises one or more high dose active pharmaceutical ingredients in an amount of 250-600 mg in the flow pack.

89. The flowpack according to any one of the preceding claims, wherein the one or more distinct portions of particles of the first type of particles comprising the one or more high dose active pharmaceutical ingredients is present in an amount of 30-70% by weight of the population of particles.

90. The flowpack according to any one of the preceding claims, wherein the one or more distinct portions of particles of the first type of particles comprising the one or more high dose active pharmaceutical ingredients is present in an amount of 40-60% by weight of the population of particles.

91. The flowpack according to any one of the preceding claims, wherein a series of at least 5 samples of the population of particles each having the same fixed weight in the range of 0.5-2 g comprises the one or more active pharmaceutical ingredients in an amount varying with a relative standard deviation (RSD) below 10%.

92. The flowpack according to any one of the preceding claims, wherein a series of at least 5 samples of the population of particles each having the same fixed weight in the range of 0.5-2 g comprises the one or more active pharmaceutical ingredients in an amount varying with a relative standard deviation (RSD) below 5%.

93. The flowpack according to any one of the preceding claims, wherein a series of at least 5 samples of the population of particles each having the same fixed weight in the range of 0.5-2 g comprises the one or more active pharmaceutical ingredients in an amount varying with a relative standard deviation (RSD) below 2%.

94. The flowpack according to any one of the preceding claims, wherein the first, second and third type of particles have an average particle size difference of at most 200 microns.

95. The flowpack according to any one of the preceding claims, wherein the first, second and third type of particles have an average particle size difference of at most 100 microns.

96. The flowpack according to any one of the preceding claims, wherein the first, second and third type of particles have an average particle size difference of at most 50 microns.

97. The flowpack according to any one of the preceding claims, wherein the one or more distinct portions of particles of the first type of particles have an average particle size difference of at most 100 microns.

98. The flowpack according to any one of the preceding claims, wherein the one or more distinct portions of particles of the first type of particles have an average particle size difference of at most 50 microns.

99. The flowpack according to any one of the preceding claims, wherein the first type of particles is present in an amount of 2-60% by weight of the population of particles.

100. The flowpack according to any one of the preceding claims, wherein the one or more pharmaceutical ingredients is microencapsulated.

101. The flowpack according to any one of the preceding claims, wherein the one or more pharmaceutical ingredients is granulated with the carrier.

102. The flowpack according to any one of the preceding claims, wherein the carrier for the one or more pharmaceutical ingredients comprises microcrystalline cellulose and starch, such as pregelatinized starch.

103. The flowpack according to any one of the preceding claims, wherein the carrier for the one or more pharmaceutical ingredients comprises one or more sugar alcohols selected from the group consisting of erythritol, maltitol, xylitol, isomalt, lactitol, mannitol, sorbitol, and combinations thereof.

104. The flowpack according to any one of the preceding claims, wherein the carrier for the one or more pharmaceutical ingredients comprises one or more binders from the group consisting of hydroxypropyl cellulose (HPC), hydroxypropylmethyl cellulose (HPMC), carboxymethyl cellulose (CMC), low substituted hydroxypropyl cellulose (L-HPC), polyvinylpyrrolidone (PVP), copovidone, and any mixture and combination thereof.

105. The flowpack according to any one of the preceding claims, further comprising a disintegrant.

106. The flowpack according to any one of the preceding claims, further comprising a disintegrant selected from the group consisting of sodium croscarmellose, crospovidone, sodium starch glycolate, and combinations thereof.

107. The flowpack according to any one of the preceding claims, further comprising one or more high-intensity sweeteners.

108. The flowpack according to any one of the preceding claims, further comprising calcium carbonate or talc.

109. The flowpack according to any one of the preceding claims, further comprising a glidant.

110. The flowpack according to any one of the preceding claims, further comprising a glidant selected from the group consisting of colloidal silicon dioxide, starch, talc, tribasic calcium phosphate, magnesium stearate, magnesium carbonate, magnesium silicate, silica derivatives, and combinations thereof.

111. The flowpack according to any one of the preceding claims, further comprising a buffering agent.

112. The flowpack according to any one of the preceding claims, further comprising an effervescence system of a base and an acid.

113. The flowpack according to any one of the preceding claims comprising an outer package material enclosing the population of particles.

114. The flowpack according to any one of the preceding claims comprising an outer aluminum package material enclosing the population of particles

115. The flowpack according to any one of the preceding claims comprising an outer oxygen impermeable package material enclosing the population of particles.

116. The flowpack according to any one of the preceding claims, wherein the population of particles are administered directly in the mouth.

117. The flowpack according to any one of the preceding claims, wherein the population of particles is poured into water and the water is administered in the mouth.

118. The flowpack according to any one of the preceding claims, wherein the population of particles provides an improved mouthfeel compared to a powder mixture without the second type of particles, the improved mouthfeel including at least one of less sandy mouthfeel, less dusty mouthfeel, less roughness mouthfeel, less sticky or improved texture.

119. The flowpack according to any one of the preceding claims, wherein the population of particles provides faster dissolution compared to a powder mixture without the second type of particles.

120. The flowpack according to any one of the preceding claims, wherein the population of particles provides improved taste masking compared to a powder mixture without the second type of particles.

121. The flowpack according to any one of the preceding claims, wherein the population of particles provides improved taste masking compared to an oral tablet comprising a combination of directly compressible (DC) sugar alcohol particles and non-directly compressible (non-DC).

122. The flowpack according to any one of the preceding claims, wherein the population of particles provides improved taste masking compared to an oral tablet comprising a combination of directly compressible (DC) sugar alcohol particles and non-directly compressible (non-DC) in a ratio of 0.2 and 1.2, such as 0.3 to 0.7.

123. The flowpack according to any one of the preceding claims, wherein the population of particles has a unit weight of 200 to 500 mg in the flow pack.

124. The flowpack according to any one of the preceding claims, wherein the population of particles has a unit weight of 500 to 2000 mg in the flow pack.

125. The flowpack according to any one of the preceding claims, wherein the population of particles has a unit weight of 700 to 2000 mg in the flow pack.

126. The flowpack according to any one of the preceding claims, wherein the population of particles has a unit weight of 1000 to 2000 mg in the flow pack.

127. A flowpack for oral delivery of active ingredients comprising a population of particles, an outer package material enclosing the population of particles, the population of particles comprising:
a first type of particles comprising one or more distinct portions of particles with a content of one or more active pharmaceutical ingredients and a carrier for the one or more active pharmaceutical ingredients;
a second type of particles in an amount of 30-95% by weight of the population of particles separate from the first type of particles, the second type of particles comprising at least 95% by weight of one or more sweeteners; and
a third type of particles in an amount of 1-10% by weight of the population of particles separate from the first and second type of particles, the third type of particles comprising flavor.

128. Use of a flowpack according to any of the preceding claims as a powder delivery system for improving saliva generation.

129. Use of a flowpack according to any of the preceding claims as a powder delivery system for administration of active ingredients.

130. Use of a flowpack according to any of the preceding claims as a powder delivery system for improved taste masking of active pharmaceutical ingredients.

131. A method of achieving improved taste masking of active pharmaceutical ingredients, comprising the steps of:
a) providing a flow pack comprising a population of particles according to any one of the preceding claims, the population of particles being a dry and flowable population of particles contained in an outer flowpack material,
b) resembling a liquid mouthwash by swishing said population of particles, thereby generating fluid in the oral cavity without adding water.

132. A method of achieving improved salivation, comprising the steps of:
a) providing a flow pack comprising a population of particles according to any one of the preceding claims, the population of particles being a dry and flowable population of particles contained in an outer flowpack material,
b) resembling a liquid mouthwash by swishing said population of particles, thereby generating fluid in the oral cavity without adding water.

133. A method of achieving improved salivation, comprising the steps of:
a) providing a flow pack comprising a population of particles according to any one of preceding claims, the population of particles being a dry and flowable population of particles contained in an outer flowpack material,
b) subjecting the population of particles into water, thereby obtaining an at least partly dissolved swishable powder delivery system, and
c) swishing the at least partly dissolved swishable powder delivery system, thereby generating fluid in the oral cavity.
